(19) United States
(12) Patent Application Publication
(10) Pub. No.: US 2007/0010734 A1
(43) Pub. Date: Jan. 11, 2007

(54) ELECTROMAGNETIC RESONANT CIRCUIT SLEEVE FOR IMPLANTABLE MEDICAL DEVICE

(75) Inventors: Robert W. Gray, Rochester, NY (US); Stuart G. MacDonald, Pultneyville, NY (US); Andreas Melzer, Mulheim-Ruhr (DE)

Correspondence Address: BASCH & NICKERSON LLP, 1777 PENFIELD ROAD, PENFIELD, NY, 14526, US

(73) Assignee: Biophan Technologies, Inc., West Henrietta, NY (US)

(21) Appl. No.: 11/419,277

(22) Filed: May 19, 2006

Related U.S. Application Data
(60) Provisional application No. 60/682,455, filed on May 19, 2005.
(60) Provisional application No. 60/736,584, filed on Nov. 14, 2005.

(51) Int. Cl. A61B 5/05; A61M 25/00; A61B 6/00

(52) U.S. Cl. 600/411; 600/434; 600/435

(57) ABSTRACT

A medical device enables effective magnetic resonance imaging inside a lumen of a medical device. The medical device includes a plurality of conductive traces formed on a substrate. The conductive traces form an inductive-capacitance circuit or a resistive-inductive-capacitance circuit. The inductive-capacitance circuit or resistive-inductive-capacitance circuit is tuned to a frequency associated with magnetic resonance imaging, an operating frequency associated with a magnetic resonance imaging scanner, a harmonic of an operating frequency associated with a magnetic resonance imaging scanner, or a sub-harmonic of an operating frequency associated with a magnetic resonance imaging scanner.

### PRIORITY INFORMATION

This application claims priority from U.S. Provisional Patent Application, Ser. No. 60/682,455, filed on May 19, 2005 and U.S. Provisional Patent Application Ser. No. 60/736,584, filed on Nov. 14, 2005. The entire contents of U.S. Provisional Patent Application Ser. No. 60/682,455, filed on May 19, 2005 and U.S. Provisional Patent Application Ser. No. 60/736,584, filed on Nov. 14, 2005 are hereby incorporated by reference.

### FIELD OF THE PRESENT INVENTION

The present invention is directed to a stent sleeve. More particularly, the present invention is directed to a stent sleeve that is a resonator for magnetic resonance imaging inside the stent.

### BACKGROUND OF THE PRESENT INVENTION

Stents have been implanted in vessels, ducts, or channels of the human body to act as a scaffolding to maintain the patency of the vessel, duct, or channel lumen. A drawback of stenting is the body's natural defensive reaction to the implant of a foreign object. In many patients, the reaction is characterized by a traumatic proliferation of tissue as intimal hyperplasia at the implant site, and, where the stent is implanted in a blood vessel such as a coronary artery, formation of thrombi which become attached to the stent.

Each of these adverse effects contributes to restenosis-a re-narrowing of the vessel lumen-to compromise the improvements that resulted from the initial re-opening of the lumen by implanting the stent. Consequently, a great number of stent implant patients must undergo another angiogram, on average about six months after the original implant procedure, to determine the status of tissue proliferation and thrombosis in the affected lumen. If re-narrowing has occurred, one or more additional procedures are required to stem or reverse its advancement.

Due to the drawbacks mentioned above, the patency of the vessel lumen and the extent of tissue growth within the lumen of the stent need to be examined and analyzed, and the blood flow therethrough needs to be measured, from time to time, as part of the patient's routine post-procedure examinations.

Current techniques employed magnetic resonance imaging (MRI) to visualize internal features of the body if there is no magnetic resonance distortion. However, using magnetic resonance imaging techniques to visualize implanted stents composed of ferromagnetic or electrically conductive materials is difficult because these materials cause sufficient distortion of the magnetic resonance field to preclude imaging the interior of the stent. This effect is attributable to their Faradaic physical properties in relation to the electromagnetic energy applied during the magnetic resonance imaging process.

One conventional solution to this problem is to design a stent that includes a mechanically supportive tubular structure composed primarily of metal having relatively low magnetic susceptibility, and one electrically conductive layer overlying a portion of the surface of the tubular structure to enhance properties of the stent for magnetic resonance imaging of the interior of the lumen of the stent when implanted in the body. An electrically insulative layer resides between the surface of the tubular structure of the stent and the electrically conductive layer. The tubular structure with overlying electrically conductive layer and electrically insulative layer sandwiched therebetween are arranged in a composite relationship to form an LC circuit at the desired frequency of magnetic resonance. The electrically conductive layer has a geometric formation arranged on the tubular scaffolding of the stent to function as an electrical inductance element and an electrical capacitance element.

Although the proposed solution may provide a stent structure that enables imaging and visualization of the inner lumen of an implanted stent by means of a magnetic resonance imaging technique, the actual structure of the stent that provides the imaging and visualization of the inner lumen of an implanted stent is dependent upon the actual structure of the stent. Thus, the stent must be designed in a particular manner to interactive with the overlying layer to provide a stent structure that enables imaging and visualization of the inner lumen of an implanted stent.

Therefore, it is desirable to provide a device which enables imaging and visualization of the inner lumen of an implanted stent by means of a magnetic resonance imaging technique and which is independent of the stent structure.

It is also desirable to provide a device that enables the effective designing of a stent to provide scaffolding so as to maintain the patency of the vessel, duct or channel lumen without having to design features into the stent to enable imaging and visualization of the inner lumen of an implanted stent by means of an magnetic resonance imaging technique.

### SUMMARY OF THE PRESENT INVENTION

One aspect of the present invention is a device for enabling effective magnetic resonance imaging inside a lumen of a medical device. The device includes a substrate and a plurality of conductive traces formed on the substrate, the conductive traces forming an inductive-capacitance circuit, the inductive-capacitance circuit being tuned to a frequency associated with magnetic resonance imaging.

Another aspect of the present invention is an implantable medical device. The implantable medical device includes a stent; a substrate surrounding a portion of the stent; and a plurality of conductive traces formed on the substrate, the conductive traces forming an inductive-capacitance circuit, the inductive-capacitance circuit being tuned to a frequency such that an effective resonance frequency of the stent, inductive-capacitance circuit, and surrounding in vitro conditions is substantially equal to a frequency associated with magnetic resonance imaging.

Another aspect of the present invention is a device for enabling effective magnetic resonance imaging inside a lumen of a medical device having an expandable substantially cylindrical substrate having an axial closed end and an axial open end, the axial closed end being within the axial open end; a dielectric material formed on a portion of the expandable substantially cylindrical substrate; and a plurality of conductive traces formed on the dielectric material and the expandable substantially cylindrical substrate, the conductive traces forming a variable inductive-capacitance circuit.

Another aspect of the present invention is a device for enabling effective magnetic resonance imaging inside a lumen of a medical device having a stent; an expandable substantially cylindrical substrate surrounding a portion of the stent, the expandable substantially cylindrical substrate having an axial closed end and an axial open end, the axial closed end being within the axial open end; a dielectric material formed on a portion of the substantially cylindrical substrate; and a plurality of conductive traces formed on the dielectric material and the expandable substantially cylindrical substrate, the conductive traces forming a variable inductive-capacitance circuit.

Another aspect of the present invention is a method for enabling effective magnetic resonance imaging inside a lumen of a medical device, the method wrapping a substrate around a portion of the medical device, the substrate having a plurality of conductive traces formed thereon, the conductive traces forming an inductive-capacitance circuit, the inductive-capacitance circuit being tuned to a frequency associated with magnetic resonance imaging; and crimping the substrate.

Another aspect of the present invention is a method for enabling effective magnetic resonance imaging inside a lumen of a medical device, the method placing a portion of the medical device in a substantially cylindrical substrate, the substantially cylindrical substrate having an axial closed end and an axial open end, the axial closed end being within the axial open end, the substantially cylindrical substrate having a dielectric material formed on a portion of thereof and a plurality of conductive traces formed on the dielectric material and the substantially cylindrical substrate, the conductive traces forming a variable inductive-capacitance circuit; and crimping the substrate.

Another aspect of the present invention is a method for enabling effective magnetic resonance imaging inside a lumen of a medical device, the method placing a portion of the medical device in an expandable substantially cylindrical substrate, the expandable substantially cylindrical substrate having an axial closed end and an axial open end, the axial closed end being within the axial open end, the expandable substantially cylindrical substrate having a dielectric material formed on a portion of thereof and a plurality of expandable conductive traces formed on the dielectric material and the substantially cylindrical substrate, the expandable conductive traces forming a variable inductive-capacitance circuit; and crimping the substrate.

Another aspect of the present invention is a device for enabling effective magnetic resonance imaging inside a lumen of a medical device having a substrate and a plurality of conductive traces formed on the substrate, a first portion of the conductive traces forming an inductive coil, a second portion of the conductive traces overlapping a third portion of the conductive traces with a dielectric material formed at the overlapping of and between the second portion of the conductive traces with the third portion of the conductive traces, the dielectric material and overlapped portions of the conductive traces forming a capacitor; the inductive coil and the capacitor being tuned to a frequency associated with magnetic resonance imaging.

Another aspect of the present invention is an implantable medical device having a stent; a substrate surrounding a portion of the stent; and a plurality of conductive traces formed on the substrate, a first portion of the conductive traces forming an inductive coil, a second portion of the conductive traces overlapping a third portion of the conductive traces with a dielectric material formed at the overlapping of and between the second portion of the conductive traces with the third portion of the conductive traces, the dielectric material and overlapped portions of the conductive traces forming a capacitor; the inductive coil and the capacitor being tuned to a frequency associated with magnetic resonance imaging.

Another aspect of the present invention is a device for enabling effective magnetic resonance imaging inside a lumen of a medical device having a substrate and a plurality of conductive traces formed on the substrate; the plurality of conductive traces forming a plurality of loops to create a single spiraling coil, adjacent loops of the single spiraling coil having a non-conductive material therebetween; the single spiraling coil forming an inductive coil; the adjacent loops of the single spiraling coil having a nonconductive material therebetween forming a capacitor; the inductive coil and the capacitor being tuned to a frequency associated with magnetic resonance imaging.

Another aspect of the present invention is an implantable medical device having a stent; a substrate surrounding a portion of the stent; and a plurality of conductive traces formed on the substrate; the plurality of conductive traces forming a plurality of loops to create a single spiraling coil, adjacent loops of the single spiraling coil having a nonconductive material therebetween; the single spiraling coil forming an inductive coil; the adjacent loops of the single spiraling coil having a nonconductive material therebetween forming a capacitor; the inductive coil and the capacitor being tuned to a frequency associated with magnetic resonance imaging.

Another aspect of the present invention is a device for enabling effective magnetic resonance imaging inside a lumen of a medical device having a substantially cylindrical substrate; a first plurality of conductive traces formed on the substantially cylindrical substrate; and a second plurality of conductive traces formed on the substantially cylindrical substrate; the first plurality of conductive traces forming a first inductive coil having two overlapping ends with a non-conductive material therebetween, the two overlapping ends with a non conductive material therebetween forming a first capacitor; the second plurality of conductive traces forming a second inductive coil having two overlapping ends with a non-conductive material therebetween, the two overlapping ends with a non-conductive material therebetween forming a second capacitor; the first inductive coil and the second inductive coil being approximately orthogonally oriented on the substantially cylindrical substrate; the first inductive coil and the first capacitor being tuned to a first frequency associated with magnetic resonance imaging; the second inductive coil and the second capacitor being tuned to a first frequency associated with magnetic resonance imaging.

Another aspect of the present invention is an implantable medical device, comprising: a stent; a substantially cylindrical substrate surrounding a portion of the stent; a first plurality of conductive traces formed on the substantially cylindrical substrate; and a second plurality of conductive traces formed on the substantially cylindrical substrate; the first plurality of conductive traces forming a first inductive coil having two overlapping ends with a non-conductive material therebetween, the two overlapping ends with a non-conductive material therebetween forming a first capacitor; the second plurality of conductive traces forming a second inductive coil having two overlapping ends with a non-conductive material therebetween, the two overlapping ends with a non-conductive material therebetween forming a second capacitor; the first inductive coil and the second inductive coil being approximately orthogonally oriented on the substantially cylindrical substrate; the first inductive coil and the first capacitor being tuned to a first frequency associated with magnetic resonance imaging; the second hductive coil and the second capacitor being tuned to a first frequency associated with magnetic resonance imaging.

Another aspect of the present invention is a device for enabling effective magnetic resonance imaging inside a lumen of a medical device having a substantially cylindrical substrate; a first plurality of conductive traces formed on the substantially cylindrical substrate; and a second plurality of conductive traces formed on the substantially cylindrical substrate; the first plurality of conductive traces forming a first plurality of loops to create a first spiraling inductive coil having two overlapping ends with a nonconductive material therebetween, adjacent loops of the first spiraling coil having a non-conductive material therebetween, the two overlapping ends with a non-conductive material therebetween and adjacent loops of the first spiraling coil having a non-conductive material therebetween forming a first capacitor; the second plurality of conductive traces forming a second plurality of loops to create a second spiraling inductive coil having two overlapping ends with a nonconductive material therebetween, adjacent loops of the second spiraling coil having a non-conductive material therebetween, the two overlapping ends with a non-conductive material therebetween and adjacent loops of the second spiraling coil having a non-conductive material therebetween forming a second capacitor; the first spiraling inductive coil and the second spiraling inductive coil being approximately orthogonally oriented on the substantially cylindrical substrate; the first spiraling inductive coil and the first capacitor being tuned to a first frequency associated with magnetic resonance imaging; the second spiraling inductive coil and the second capacitor being tuned to a first frequency associated with magnetic resonance imaging.

Another aspect of the present invention is an implantable medical device having a stent; a substantially cylindrical substrate surrounding a portion of the stent; a first plurality of conductive traces formed on the substantially cylindrical substrate; and a second plurality of conductive traces formed on the substantially cylindrical substrate; the first plurality of conductive traces forming a first plurality of loops to create a first spiraling inductive coil having two overlapping ends with a non-conductive material therebetween, adjacent loops of the first spiraling coil having a non-conductive material therebetween, the two overlapping ends with a non-conductive material therebetween and adjacent loops of the first spiraling coil having a nonconductive material therebetween forming a first capacitor; the second plurality of conductive traces forming a second plurality of loops to create a second spiraling inductive coil having two overlapping ends with a non-conductive material therebetween, adjacent loops of the second spiraling coil having a non-conductive material therebetween, the two overlapping ends with a non-conductive material therebetween and adjacent loops of the second spiraling coil having a nonconductive material therebetween forming a second capacitor; the first spiraling inductive coil and the second spiraling inductive coil being approximately orthogonally oriented on the substantially cylindrical substrate; the first spiraling inductive coil and the first capacitor being tuned to a first frequency associated with magnetic resonance imaging; the second spiraling inductive coil and the second capacitor being tuned to a first frequency associated with magnetic resonance imaging.

### BRIEF DESCRIPTION OF THE DRAWINGS

The present invention may take form in various components and arrangements of components, and in various steps and arrangements of steps. The drawings are only for purposes of illustrating a preferred embodiment or embodiments and are not to be construed as limiting the present invention, wherein:

FIG. 1 shows a sleeve substrate having a resonance coil formed thereon according to the concepts of the present invention;

FIG. 2 shows the wrapping of the sleeve substrate of FIG. 1 according to the concepts of the present invention;

FIG. 3 shows the crimped sleeve substrate wrapped around a collapsed stent according to the concepts of the present invention;

FIG. 4 illustrates a manufacturing web transporting a number of sleeve substrates according to the concepts of the present invention;

FIG. 5 a sleeve substrate having a resonance coil formed using a folding routine according to the concepts of the present invention;

FIG. 6 shows the sleeve substrate of FIG. 5 prior to folding according to the concepts of the present invention;

FIG. 7 shows a manufacturing device for forming the resonance coil upon a substrate;

FIG. 8 shows another embodiment of a sleeve substrate having a resonance coil and variable capacitance formed thereon according to the concepts of the present invention;

FIG. 9 shows the wrapping of the sleeve substrate of FIG. 8 according to the concepts of the present invention;

FIG. 10 shows another embodiment of a sleeve substrate having a resonance coil and variable capacitance formed thereon according to the concepts of the present invention;

FIG. 11 shows the wrapping of the sleeve substrate of FIG. 10 according to the concepts of the present invention;

FIG. 12 shows another embodiment of a sleeve substrate having a resonance coil formed thereon according to the concepts of the present invention;

FIG. 13 shows another embodiment of a sleeve substrate having a resonance coil and non-linear variable capacitance formed thereon according to the concepts of the present invention;

FIG. 14 shows a sleeve substrate formed around a stent according to the concepts of the present invention;

FIG. 15 shows another embodiment of a sleeve substrate having a resonance coil and non-linear variable capacitance formed thereon according to the concepts of the present invention;

FIG. 16 is an expanded view of the traces showing the resonance coil construction

FIG. 17 shows a sleeve substrate having multiple resonance coils formed thereon according to the concepts of the present invention;

FIG. 18 shows another embodiment of a sleeve substrate having multiple resonance coils and variable capacitance formed thereon according to the concepts of the present invention;

FIG. 19 shows the wrapping of the sleeve substrate of FIG. 18 according to the concepts of the present invention;

FIG. 20 shows a sleeve substrate having a resonance coil with multiple (stacked) loops formed thereon according to the concepts of the present invention;

FIG. 21 shows a side perspective of the sleeve substrate having a resonance coil with multiple (stacked) loops formed thereon illustrated by FIG. 20 according to the concepts of the present invention

FIG. 22 illustrates a stent assembly according to the concepts of the present invention;

FIG. 23 illustrates resonant circuits on a cylinder membrane according to the concepts of the present invention;

FIG. 24 illustrates a stent sleeve assembly according to the concepts of the present invention;

FIG. 25 illustrates circuits on a flat film membrane wrapped around a stent according to the concepts of the present invention;

FIG. 26 illustrates forming circuits on a membrane according to the concepts of the present invention;

FIG. 27 illustrates a side view of the stent circuit assembly according to the concepts of the present invention; and

FIG. 28 illustrates a substrate having a resonance coil with multiple (non-stacked) loops formed thereon according to the concepts of the present invention.

### DETAILED DESCRIPTION OF THE PRESENT INVENTION

The present invention will be described in connection with preferred embodiments; however, it will be understood that there is no intent to limit the present invention to the embodiments described herein. On the contrary, the intent is to cover all alternatives, modifications, and equivalents as may be included within the spirit and scope of the present invention as defined by the appended claims.

For a general understanding of the present invention, reference is made to the drawings. In the drawings, like reference numbering has been used throughout to designate identical or equivalent elements. It is also noted that the various drawings illustrating the present invention may not have been drawn to scale and that certain regions may have been purposely drawn disproportionately so that the features and concepts of the present invention could be properly illustrated.

As noted above, the present invention is directed to a device which enables imaging and visualization of the inner lumen of an implanted stent by means of an magnetic resonance imaging technique and which is independent of the stent structure and/or a device that enables the effective designing of a stent to provide scaffolding so as to maintain the patency of the vessel, duct or channel lumen without having to design features into the stent to enable imaging and visualization of the inner lumen of an implanted stent by means of an magnetic resonance imaging technique.

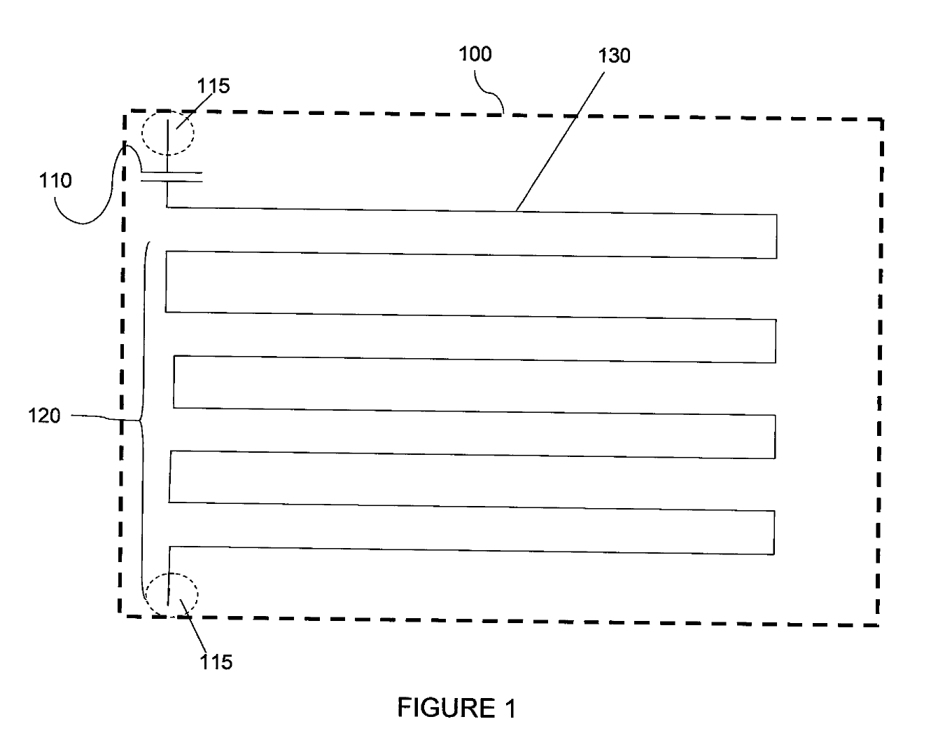

As illustrated in FIG. 1, a substrate 100 has formed thereon conductive traces 130, composed of film coatings of metal or any thin pliable conductive material. The traces 130 are formed so as to create a resonance coil or coils 120 that will be used in forming a LC circuit that is tuned to the desired frequency of magnetic resonance imaging or other desired frequency It is noted that the traces 130 may also be formed so as to create a resonance coil or coils 120 that will be used in forming a RLC circuit that is tuned to the desired frequency of magnetic resonance imaging or other desired frequency.

In this embodiment, the “resistor” is the “conductive” material or conductive traces 130. The resistor value is controlled by the dimensions of the conductor as well as the material selected for the conductor. Also, the material for the conductor may vary along the length of the tracing forming the inductor, thereby providing a resistive parameter to the circuit.

The degree of resonance or ‘Q’ of either the formed LC or formed RLC circuit is a degree of resonance at the Lamar frequency of the magnetic resonance imaging system or the desired resonance frequency to permit clinically effective imaging inside the lumen of the stent. It is noted that this is the frequency of the system as deployed; e.g. in vitro;, not the frequency in air.

The substrate 100 may, optionally, include a nominal capacitor 110 to provide a minimum capacitance for the LC or RLC circuit that is tuned to desired frequency of magnetic resonance imaging or other desired frequency.

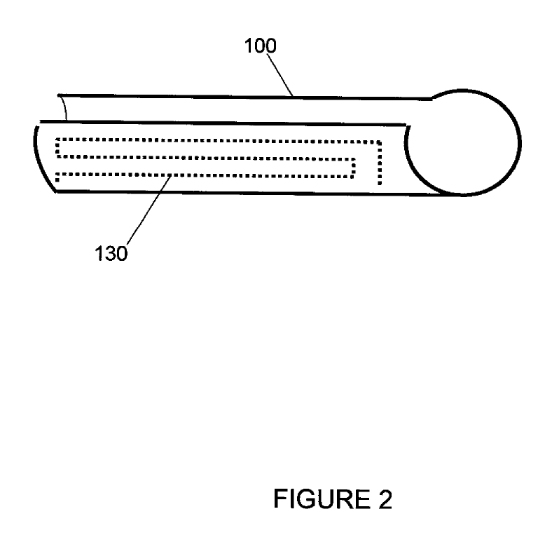

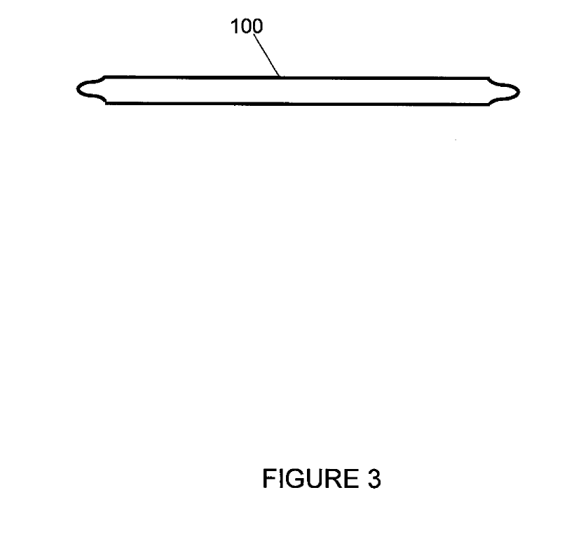

The substantial portion of the capacitance may be realized by the capacitance between the traces 130 in region 115 when the substrate 100 is wrapped into a substantially cylinder shape, as illustrated in FIG. 2, to form a sleeve. The substrate 100 can be wrapped around a medical device as illustrated in FIG. 3. When surrounding a medical device, the traces 130 are insulated by an insulative dielectric material (not shown) so that when the traces 130 in region 115 overlap, due to the wrapping of the substrate 100 as illustrated in FIG. 2, the overlapped portions of the traces 130 form a capacitor. The capacitance of the trace formed capacitor in region 115 is variable as the wrapping of the substrate 100 becomes tighter (contracts) or is loosened (expands).

As noted above, the stent must enable imaging and visualization of the inner lumen of an implanted stent by means of a magnetic resonance imaging technique, thus the stent must have an associated resonance circuit that is tuned to the desired frequency of magnetic resonance. The substrate sleeve of FIG. 1 provides the resonance circuit that may be tuned to the desired frequency of magnetic resonance or other desired frequency, independent of the stent.

To be in resonance, the resonance circuit of the substrate sleeve of FIG. 1 must include an LC or RLC circuit that is tuned to the desired frequency of magnetic resonance or other desired frequency In this embodiment, the traces 130 are formed to create the inductive properties and the overlapping of the traces, when the sleeve is wrapped, creates the capacitive properties. Again, it is noted that a resistive value related to the dimensions of the conductor as well as the material selected for the conductor may be included in the resonance circuit of the substrate sleeve.

It is noted that as the wrapping of the substrate 100 becomes tighter (contracts), the overall inductance of the resonance circuit of the substrate sleeve decreases, but the overall capacitance of the resonance circuit of the substrate sleeve increases because the area of the overlapping trace portions becomes greater, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that as the wrapping of the substrate 100 becomes looser (expands), the overall inductance of the resonance circuit of the substrate sleeve increases, but the overall capacitance of the resonance circuit of the substrate sleeve decreases because the area of the overlapping trace portions becomes lesser, thereby substantially maintaining resonance with the desired frequency of magnetic resonance imaging or other desired frequency.

It is noted that the combination of the increasing of the overall inductance of the resonance circuit of the substrate sleeve and the decreasing of the overall capacitance of the resonance circuit of the substrate sleeve may also enable the maintaining of resonance within a desired bandwidth, which may or may not be in resonance with the magnetic resonance imaging scanner.

The substrate 100 may be a biodegradable substrate that essentially decomposes once the stent is positioned in the body. It is further noted that the substrate 100 may be thermally degradable, chemically degradable, and/or optically degradable. The substrate 100 may also include drugs or medical agents that are therapeutically released upon the decomposition of the substrate. Lastly, the substrate 100 and included resonance circuit are expandable without resulting in breakage. It is noted that the substrate or support web 100, may be biodegradable and may have adhesive properties useful during manufacture and implantation; however, after biodegradation, the applied conductive traces 130 retain an electrically insulating coating or sheath that prevents unwanted shorting even under repeated flexing of the stent/circuit device in the body.

As discussed above, the substrate sleeve is wrapped, more particularly; the substrate sleeve is wrapped around a stent and crimped, as illustrated in FIG. 3, to form a stent device with an independent resonance circuit. The resonance circuit can be designed to complement the resonance frequency of an implanted stent so that the combination of the resonance circuit, the implanted stent, and surrounding environmental conditions has an effective resonance frequency that is substantially equal to the operating frequency of the magnetic resonance imaging scanner. It is noted that the resonance circuit can be designed to complement the resonance frequency of any implanted device having a lumen to be imaged so that the combination of the resonance circuit, the implanted device, and surrounding environmental conditions has an effective resonance frequency that is substantially equal to the operating frequency of the magnetic resonance imaging scanner. Moreover, the resonance circuit need not be designed to interact with the conductive material of the stent to provide resonance, but merely needs to be designed to contemplate the degree of expansion of the stent so that the proper inductance can be generated with the coil formations and the proper capacitance can be generated with the trace overlap.

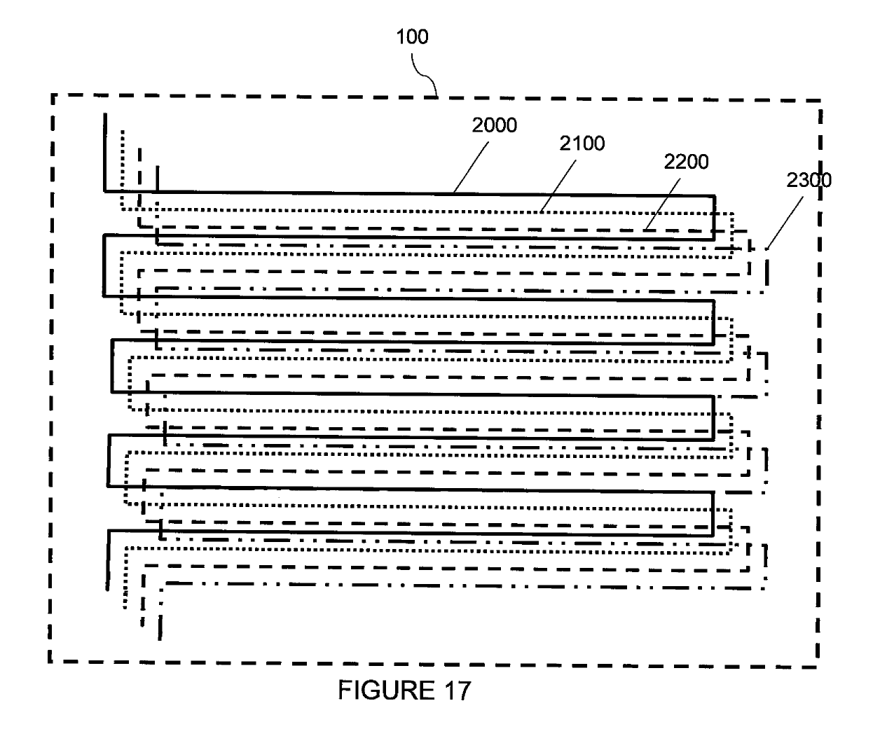

As illustrated in FIG. 17, a substrate 100 has formed thereon conductive traces (2000 2100, 2200, and 2300) composed of film coatings of metal or any thin pliable conductive material. The traces are formed so as to create independent resonance coils tuned to different frequencies. It is noted that these frequencies may be harmonics. The coils are formed by the traces running on top of each other with an insulating material therebetween. It is noted that the insulating material may be a dielectric to provide capacitance.

The conductive traces (2000 2100, 2200, and 2300) are used in forming a LC circuit that is tuned to the desired frequency of magnetic resonance imaging or other desired frequency It is noted that the traces may also be formed so as to create independent resonance coils that will be used in forming a RLC circuit that is tuned to the desired frequency of magnetic resonance imaging or other desired frequency.

In this embodiment, the “resistor” is the “conductive” material or conductive traces. The resistor value is controlled by the dimensions of the conductor as well as the material selected for the conductor. Also, the material for the conductor may vary along the length of the tracing forming the inductor, thereby providing a resistive parameter to the circuit.

The degree of resonance or ‘Q’ of either the formed LC or formed RLC circuit is a degree of resonance at the Lamar frequency of the magnetic resonance imaging system to permit clinically effective imaging inside the lumen of the stent.

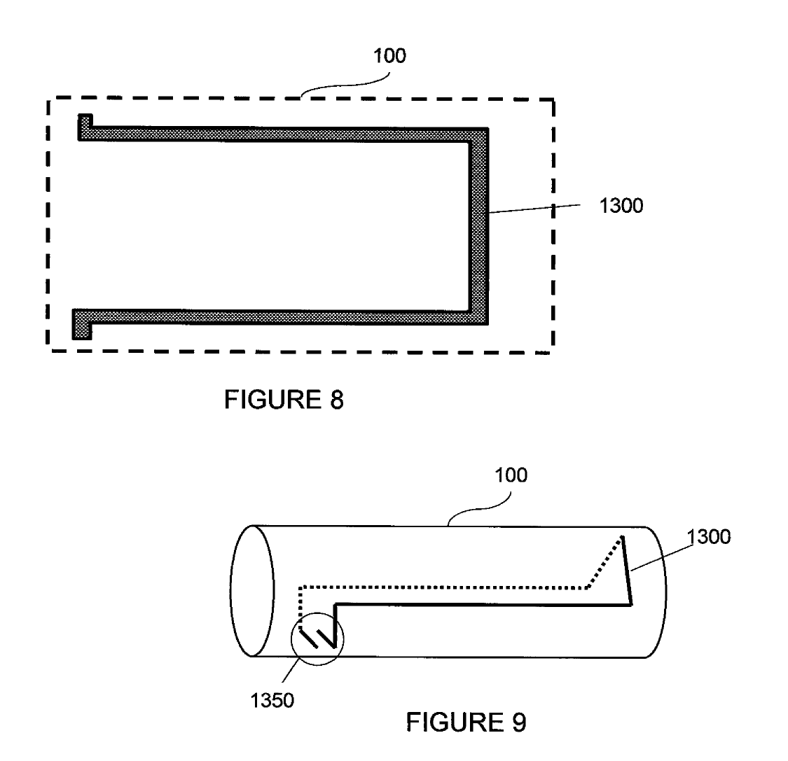

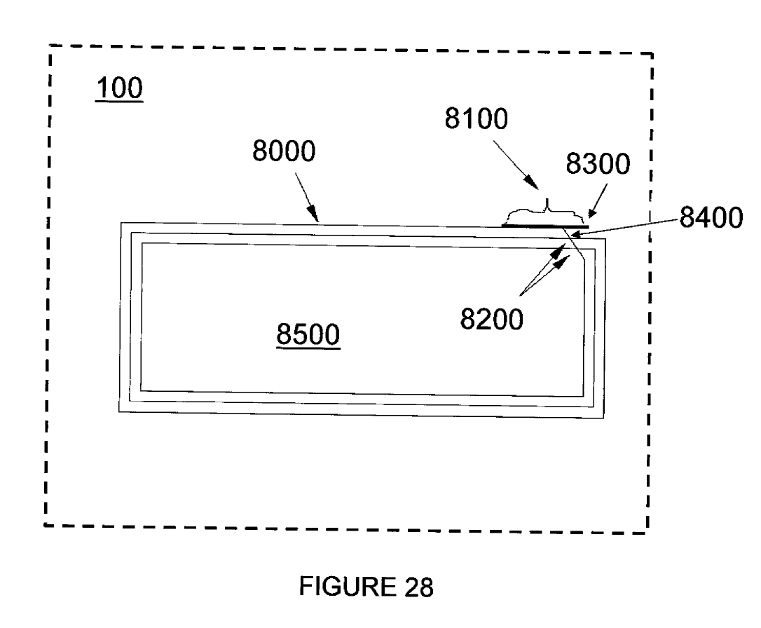

As illustrated in FIG. 8, a substrate 100 has formed thereon a conductive trace 1300, composed of film coating of metal or any thin pliable conductive material. The trace 1300 is formed so as to create a single resonance coil that will be used in forming a LC circuit that is tuned to the desired frequency of magnetic resonance imaging or other desired frequency It is noted that the trace 1300 can be formed so as to create a single resonance coil of a multi-loop inductor coil, as illustrated in FIG. 28, wherein the multi-loop inductor coil will be used in forming a LC circuit that is tuned to the desired frequency of magnetic resonance imaging or other desired frequency It is further noted that the traces 1300 may also be formed so as to create a resonance coil that will be used in forming a RLC circuit that is tuned to the desired frequency of magnetic resonance imaging or other desired frequency Also, it is noted that the traces 1300 may also be formed so as to create a single resonance coil of a multi-loop inductor coil, as illustrated in FIG. 28, wherein the multi-loop inductor coil will be used in forming a RLC circuit that is tuned to the desired frequency of magnetic resonance imaging or other desired frequency.

In this embodiment, the “resistor” is the “conductive” material or conductive traces 1300. The resistor value is controlled by the dimensions of the conductor as well as the material selected for the conductor. Also, the material for the conductor may vary along the length of the tracing forming the inductor, thereby providing a resistive parameter to the circuit.

The degree of resonance or ‘Q’ of either the formed LC or formed RLC circuit is a degree of resonance at the Lamar frequency of the magnetic resonance imaging system to permit clinically effective imaging inside the lumen of the stent.

The capacitance is realized by the capacitance by the overlapping of the end portions of the trace 1300 in region 1350 when the substrate 100 is wrapped into a substantially cylinder shape, as illustrated in FIG. 9, to form a sleeve. The trace 1300 is insulated by an insulative dielectric material (not shown) so that when the end portions of the trace 1300 in region 1350 overlap, due to the wrapping of the substrate 100 as illustrated in FIG. 9, the overlapped portions of the trace 1300 form a capacitor. The capacitance of the trace formed capacitor in region 1350 is variable as the wrapping of the substrate 100 becomes tighter (contracts) or is loosened (expands).

As noted above, the stent must enable imaging and visualization of the inner lumen of an implanted stent by means of a magnetic resonance imaging technique, thus the stent must have an associated resonance circuit that is tuned to the desired frequency of magnetic resonance when deployed in the patient's body or deployed in vitro. The substrate sleeve of FIGS. 8 and 9 provides the resonance circuit that is tuned to the desired frequency of magnetic resonance independent of the stent. The resonance circuit of FIGS. 8 and 9 can also be designed to complement the resonance frequency of an implanted stent so that the combination of the resonance circuit, the implanted stent, and surrounding environmental conditions has an effective resonance frequency that is substantially equal to the operating frequency of the magnetic resonance imaging scanner. It is noted that the resonance circuit can be designed to complement the resonance frequency of any implanted device having a lumen to be imaged so hat the combination of the resonance circuit, the implanted device, and surrounding environmental conditions has an effective resonance frequency that is substantially equal to the operating frequency of the magnetic resonance imaging scanner.

To be in resonance, the substrate sleeve of FIGS. 8 and 9 must include an LC or RLC circuit such that the entire implanted system is tuned to the desired frequency of magnetic resonance when deployed in a patient's body or other desired frequency.

In this embodiment, the traces 1300 are formed to create the inductive properties and the overlapping of the traces, when the sleeve is wrapped, creates the capacitive properties. Again, it is noted that a resistive value related to the dimensions of the conductor as well as the material selected for the conductor may be included in the resonance circuit of the substrate sleeve.

It is noted that as the wrapping of the substrate 100 becomes tighter (contracts), the overall inductance of the resonance circuit of the substrate sleeve decreases, but the overall capacitance of the resonance circuit of the substrate sleeve increases because the area of the overlapping trace portions becomes greater, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that as the wrapping of the substrate 100 becomes looser (expands), the overall inductance of the substrate sleeve increases, but the overall capacitance of the substrate sleeve decreases because the area of the overlapping trace portions becomes lesser, thereby substantially maintaining resonance with the desired frequency of magnetic resonance imaging or other desired frequency.

It is noted that the combination of the increasing of the overall inductance of the resonance circuit of the substrate sleeve and the decreasing of the overall capacitance of the resonance circuit of the substrate sleeve may also enable the maintaining of resonance within a desired bandwidth, which may or may not be in resonance with the magnetic resonance imaging scanner.

The substrate 100 may be a biodegradable substrate that essentially decomposes once the stent is positioned in the body. It is further noted that the substrate 100 may be thermally degradable, chemically degradable, and/or optically degradable. The substrate 100 may also include drugs or medical agents that are therapeutically released upon the decomposition of the substrate. Lastly, the substrate 100 and included resonance circuit are expandable without resulting in breakage. It is noted that the substrate or support web 100, may be biodegradable and may have adhesive properties useful during manufacture and implantation; however, after biodegradation, the applied conductive traces 1300 retain an electrically insulating coating or sheath that prevents unwanted shorting even under repeated flexing of the stent/circuit device in the body.

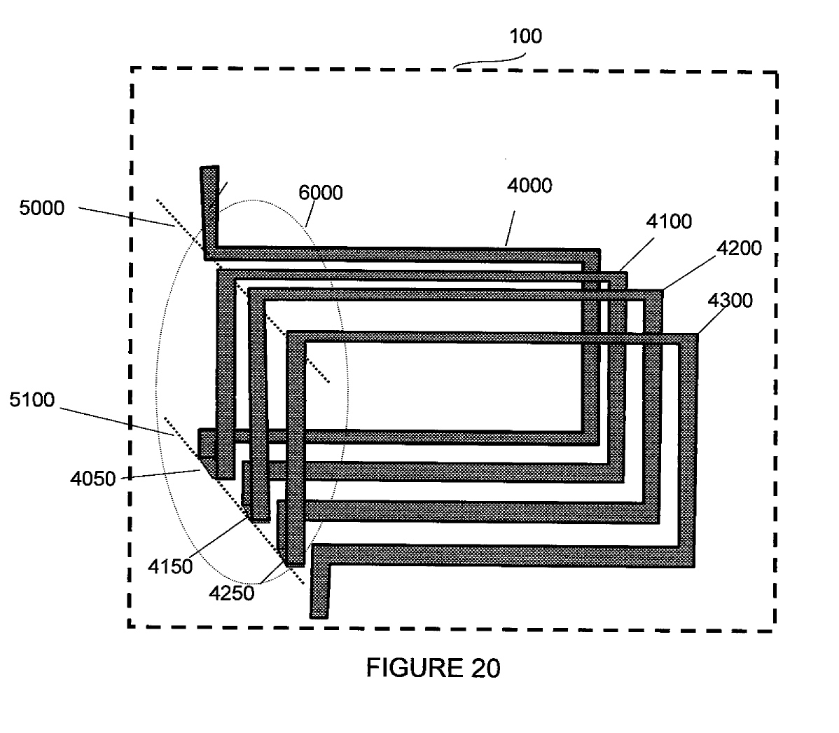

The embodiment illustrated in FIG. 8 is applicable to a resonance coil constructed of multiple or stacked loops, as illustrated in FIG. 20. In FIG. 20, a substrate 100 has formed thereon a conductive trace with stacked or multiple loops (4000, 4100, 4200, and 4300), composed of film coating of metal or any thin pliable conductive material. The trace is formed so as to create a single resonance coil having stacked or multiple loops (4000, 4100, 4200, and 4300) that will be used in forming a LC circuit that is tuned to the desired frequency of magnetic resonance imaging or other desired frequency It is noted that the trace may also be formed so as to create a resonance coil that will be used in forming a RLC circuit that is tuned to the desired frequency of magnetic resonance imaging or other desired frequency

In this embodiment, the “resistor” is the “conductive” material or conductive trace. The resistor value is controlled by the dimensions of the conductor as well as the material selected for the conductor. Also, the material for the conductor may vary along the length of the tracing forming the inductor, thereby providing a resistive parameter to the circuit.

The degree of resonance or ‘Q’ of either the formed LC or formed RLC circuit is a degree of resonance at the Lamar frequency of the magnetic resonance imaging system to permit clinically effective imaging inside the lumen of the stent.

The capacitance is realized by the capacitance by the overlapping of the end portions of the trace when the substrate 100 is wrapped into a substantially cylinder shape to form a sleeve. The trace is insulated by an insulative dielectric material (not shown) so that when the end portions of the trace overlap, due to the wrapping of the substrate 100, the overlapped portions of the trace form a capacitor. The capacitance of the trace formed capacitor is variable as the wrapping of the substrate 100 becomes tighter (contracts) or is loosened (expands).

As noted above, the stent must enable imaging and visualization of the inner lumen of an implanted stent by means of a magnetic resonance imaging technique, thus the stent must have an associated resonance circuit that is tuned to the desired frequency of magnetic resonance. The substrate sleeve provides the resonance circuit that is tuned to the desired frequency of magnetic resonance independent of the stent. The resonance circuit can also be designed to complement the resonance frequency of an implanted stent so that the combination of the resonance circuit, the implanted stent, and surrounding environmental conditions has an effective resonance frequency that is substantially equal to the operating frequency of the magnetic resonance imaging scanner. It is noted that the resonance circuit can be designed to complement the resonance frequency of any implanted device having a lumen to be imaged so that the combination of the resonance circuit, the implanted device, and surrounding environmental conditions has an effective resonance frequency that is substantially equal to the operating frequency of the magnetic resonance imaging scanner.

To be in resonance, the substrate sleeve of FIG. 20 must include an LC or RLC circuit that is tuned to the operating frequency of the magnetic resonance imaging scanner or other desired frequency.

In this embodiment, the trace has stacked or multiple loops (4000, 4100, 4200, and 4300) to create the inductive properties and the overlapping of the trace, when the sleeve is wrapped, creates the capacitive properties. Again, it is noted that a resistive value related to the dimensions of the conductor as well as the material selected for the conductor may be included in the resonance circuit of the substrate sleeve.

It is noted that as the wrapping of the substrate 100 becomes tighter (contracts), the overall inductance of the resonance circuit of the substrate sleeve decreases, but the overall capacitance of the resonance circuit of the substrate sleeve increases because the area of the overlapping trace portions becomes greater, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that as the wrapping of the substrate 100 becomes looser (expands), the overall inductance of the substrate sleeve increases, but the overall capacitance of the substrate sleeve decreases because the area of the overlapping trace portions becomes lesser, thereby substantially maintaining resonance with the desired frequency of magnetic resonance imaging or other desired frequency.

It is noted that the combination of the increasing of the overall inductance of the resonance circuit of the substrate sleeve and the decreasing of the overall capacitance of the resonance circuit of the substrate sleeve may also enable the maintaining of resonance within a desired bandwidth, which may or may not be in resonance with the magnetic resonance imaging scanner.

The substrate 100 may be a biodegradable substrate that essentially decomposes once the stent is positioned in the body. It is further noted that the substrate 100 may be thermally degradable, chemically degradable, and/or optically degradable. The substrate 100 may also include drugs or medical agents that are therapeutically released upon the decomposition of the substrate. Lastly, the substrate 100 and included resonance circuit are expandable without resulting in breakage. It is noted that the substrate or support web 100, may be biodegradable and may have adhesive properties useful during manufacture and implantation; however, after biodegradation, the applied conductive traces 1300 retain an electrically insulating coating or sheath that prevents unwanted shorting even under repeated flexing of the stent/circuit device in the body.

As illustrated in FIG. 20, end portions of the multiple or stacked loops (4000, 4100, 4200, and 4300) may be aligned along dotted lines 5000 and 5100. The multiple or stacked loops (4000, 4100, 4200, and 4300) are electrically connected to each other by conductive trace portions 4050, 4150, and 4250). More specifically, loop 4000 may be electrically connected to loop 4100 through conductive trace portion 4050; bop 4100 may be electrically connected to loop 4200 through conductive trace portion 4150; and loop 4200 may be electrically connected to loop 4300 through conductive trace portion 4250. By connecting the various loops in this fashion, an inductive coil is realized.

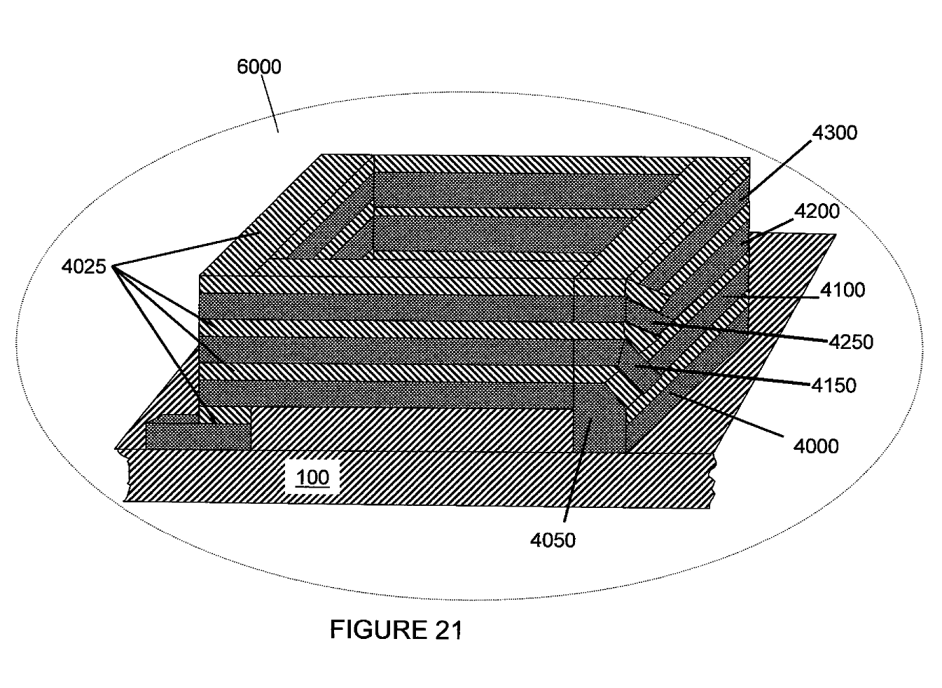

It is noted that the conductive trace portions may be replaced with a dielectric to provide a capacitive connection between the multiple or stacked loops. A better illustration of this construction is provided by FIG. 21, which illustrates a side perspective of the multiple or stacked loops at cross-section 6000 of FIG. 20.

In FIG. 21, the multiple or stacked loops 4000, 4100, 4200, and 4300) are formed on the substrate 100. Between each loop, an insulating film or layer 4025 is provided.

As illustrated in FIG. 21, loop 4000 is formed on substrate 100 and may be electrically connected to loop 4100 through conductive trace portion 4050 with an insulating film or layer 4025 between loop 4000 and loop 4100; loop 4100 may be electrically connected to loop 4200 through conductive trace portion 4150 with an insulating film or layer 4025 between loop 4100 and loop 4200; and loop 4200 may be electrically connected to loop 4300 through conductive trace portion 4250 with an insulating film or layer 4025 between loop 4200 and loop 4300. Again, by connecting the various loops in this fashion, an inductive coil is realized.

It is noted that the conductive trace portions may be replaced with a dielectric to provide a capacitive connection between the multiple or stacked loops.

It is noted that the individual loops (4000, 4100, 4200, and 4300) may be formed to have distinct shapes and areas.

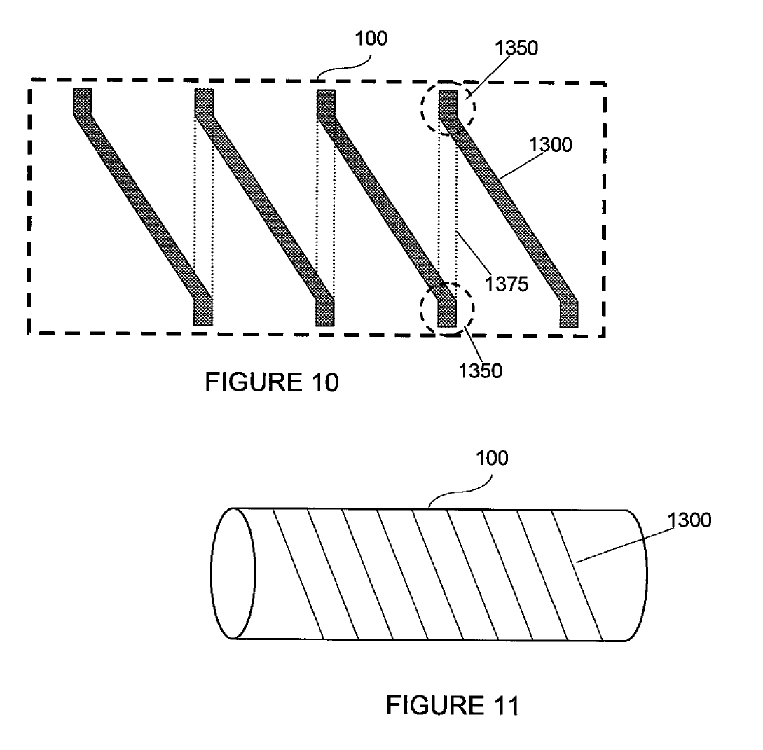

As illustrated in FIG. 10, a substrate 100 has formed thereon conductive traces 1300, composed of film coatings of metal or any thin pliable conductive material. The traces 1300 are formed so as to create a single spiraling resonance coil that will be used in forming a LC circuit that is tuned to the desired frequency of magnetic resonance imaging or other desired frequency. It is noted that the traces 1300 may also be formed so as to create a single spiraling resonance coil that will be used in forming a RLC circuit that is tuned to the desired frequency of magnetic resonance imaging or other desired frequency.

In this embodiment, the “resistor” is the “conductive” material or conductive traces 1300. The resistor value is controlled by the dimensions of the conductor as well as the material selected for the conductor. Also, the material for the conductor may vary along the length of the tracing forming the inductor, thereby providing a resistive parameter to the circuit.

The degree of resonance or ‘Q’ of either the formed LC or formed RLC circuit is a degree of resonance at the Lamar frequency of the magnetic resonance imaging system to permit clinically effective imaging inside the lumen of the stent.

The capacitance is realized by the capacitance by the overlapping of the end portions of the traces 1300 in region 1350 when the substrate 100 is wrapped into a substantially cylinder shape, as illustrated in FIG. 11, to form a sleeve. The end portions of the traces 1300 are formed so that the end portions are aligned as illustrated by dashed box 1375.

The traces 1300 are insulated by an insulative dielectric material (not shown) so that when the end portions of the traces 1300 in region 1350 overlap, due to the wrapping of the substrate 100 as illustrated in FIG. 11, the overlapped portions of the traces 1300 form a capacitor. The capacitance of the trace formed capacitor in region 1350 is variable as the wrapping of the substrate 100 becomes tighter (contracts) or is loosened (expands).

As noted above, the stent must enable imaging and visualization of the inner lumen of an implanted stent by means of a magnetic resonance imaging technique, thus the stent must have an associated resonance circuit that is tuned to the desired frequency of magnetic resonance. The substrate sleeve of FIGS. 10 and 11 provides the resonance circuit that is tuned to the desired frequency of magnetic resonance independent of the stent. The resonance circuit of FIGS. 10 and 11 can also be designed to complement the resonance frequency of an implanted stent so that the combination of the resonance circuit, the implanted stent, and surrounding environmental conditions has an effective resonance frequency that is substantially equal to the operating frequency of the magnetic resonance imaging scanner. It is noted that the resonance circuit can be designed to complement the resonance frequency of any implanted device having a lumen to be imaged so that the combination of the resonance circuit, the implanted device, and surrounding environmental conditions has an effective resonance frequency that is substantially equal to the operating frequency of the magnetic resonance imaging scanner. It is further noted that for all embodiments disclosed herein, the resonance circuits can also be designed to complement the resonance frequency of an implanted device so that the combination of the resonance circuit, the implanted device, and surrounding environmental conditions has an effective resonance frequency that is substantially equal to the operating frequency of the magnetic resonance imaging scanner. It is also noted that for all embodiments disclosed herein, the resonance circuits and the combination of the resonance circuit, the implanted device, and surrounding environmental conditions may be tuned to have an effective resonance frequency that is substantially equal to a harmonic or sub-harmonic frequency of the operating frequency of the magnetic resonance imaging scanner.

To be in resonance, the substrate sleeve of FIGS. 10 and 11 must include an LC or RLC circuit that is tuned to the desired frequency of magnetic resonance or other desired frequency.

In this embodiment, the traces 1300 are formed to create the inductive properties and the overlapping of the traces, when the sleeve is wrapped, creates the capacitive properties. Again, it is noted that a resistive value related to the dimensions of the conductor as well as the material selected for the conductor may be included in the resonance circuit of the substrate sleeve.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that the combination of the increasing of the overall inductance of the resonance circuit of the substrate sleeve and the decreasing of the overall capacitance of the resonance circuit of the substrate sleeve may also enable the maintaining of resonance within a desired bandwidth, which may or may not be in resonance with the magnetic resonance imaging scanner.

The substrate 100 may be a biodegradable substrate that essentially decomposes once the stent is positioned in the body. It is further noted that the substrate 100 may be thermally degradable, chemically degradable, and/or optically degradable. The substrate 100 may also include drugs or medical agents that are therapeutically released upon the decomposition of the substrate. Lastly, the substrate 100 and included resonance circuit are expandable without resulting in breakage. It is noted that the substrate or support web 100, may be biodegradable and may have adhesive properties useful during manufacture and implantation; however, after biodegradation, the applied conductive traces 1300 retain an electrically insulating coating or sheath that prevents unwanted shorting even under repeated flexing of the stent/circuit device in the body.

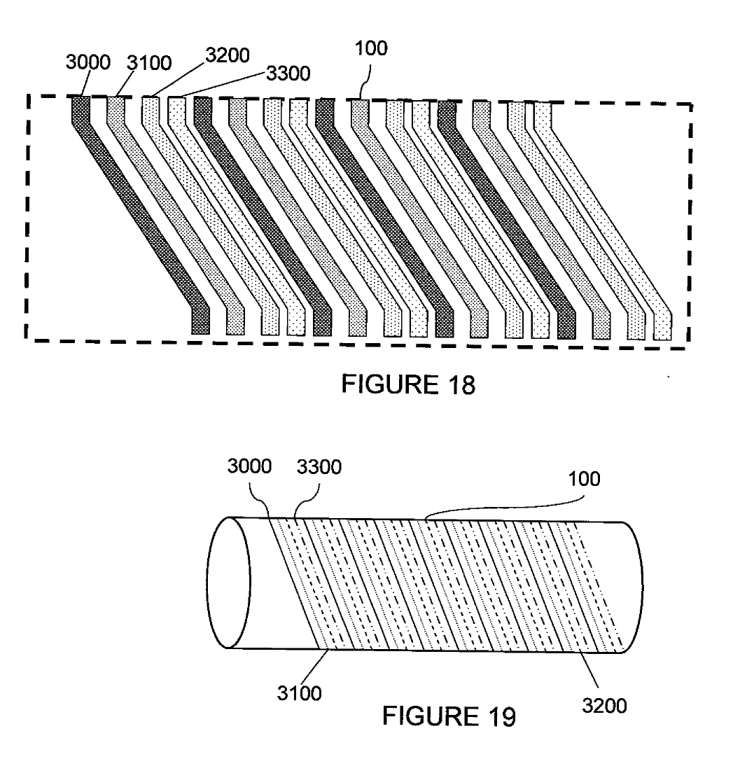

As illustrated in FIG. 18, a substrate 100 has formed thereon conductive traces P000, 3100, 3200, and 3300) composed of film coatings of metal or any thin pliable conductive material. The traces are formed so as to create independent spiraling resonance coils tuned to different frequencies. It is noted that these frequencies may be harmonics. The spiraling coils are formed by the traces running on top of each other with an insulating material therebetween. It is noted that the insulating material may be a dielectric to provide capacitance.

The conductive traces (3000 3100, 3200, and 3300) are used in forming a LC circuit that is tuned to the desired frequency of magnetic resonance imaging or other desired frequency It is noted that the traces may also be formed so as to create independent resonance coils that will be used in forming a RLC circuit that is tuned to the desired frequency of magnetic resonance imaging or other desired frequency.

In this embodiment, the “resistor” is the “conductive” material or conductive traces. The resistor value is controlled by the dimensions of the conductor as well as the material selected for the conductor. Also, the material for the conductor may vary along the length of the tracing forming the inductor, thereby providing a resistive parameter to the circuit.

The degree of resonance or ‘Q’ of either the formed LC or formed RLC circuit is a degree of resonance at the Lamar frequency of the magnetic resonance imaging system to permit clinically effective imaging inside the lumen of the stent.

The capacitance is realized by the capacitance by the overlapping of the end portions of the traces when the substrate 100 is wrapped into a substantially cylinder shape, as illustrated in FIG. 19, to form a sleeve. The end portions of the traces are formed so that the end portions are aligned.

The traces are insulated by an insulative dielectric material (not shown) so that when the end portions of the traces overlap, due to the wrapping of the substrate 100 as illustrated in FIG. 19, the overlapped portions of the traces form a capacitor. The capacitance of the trace formed capacitor is variable as the wrapping of the substrate 100 becomes tighter (contracts) or is loosened (expands).

As noted above, the stent must enable imaging and visualization of the inner lumen of an implanted stent by means of a magnetic resonance imaging technique, thus the stent must have an associated resonance circuit that is tuned to the desired frequency of magnetic resonance. The substrate sleeve of FIGS. 18 and 19 provides the resonance circuit that is tuned to the desired frequency of magnetic resonance independent of the stent. The resonance circuit of FIGS. 18 and 19 can also be designed to complement the resonance frequency of an implanted stent so that the combination of the resonance circuit, the implanted stent, and surrounding environmental conditions has an effective resonance frequency that is substantially equal to the operating frequency of the magnetic resonance imaging scanner. It is noted that the resonance circuit can be designed to complement the resonance frequency of any implanted device having a lumen to be imaged so that the combination of the resonance circuit, the implanted device, and surrounding environmental conditions has an effective resonance frequency that is substantially equal to the operating frequency of the magnetic resonance imaging scanner.

To be in resonance, the substrate sleeve of FIGS. 18 and 19 must include an LC or RLC circuit that is tuned to the desired frequency of magnetic resonance or other desired frequency.

In this embodiment, the traces are formed to create the inductive properties and the overlapping of the traces, when the sleeve is wrapped, creates the capacitive properties. Again, it is noted that a resistive value related to the dimensions of the conductor as well as the material selected for the conductor may be included in the resonance circuit of the substrate sleeve.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that the combination of the increasing of the overall inductance of the resonance circuit of the substrate sleeve and the decreasing of the overall capacitance of the resonance circuit of the substrate sleeve may also enable the maintaining of resonance within a desired bandwidth, which may or may not be in resonance with the magnetic resonance imaging scanner.

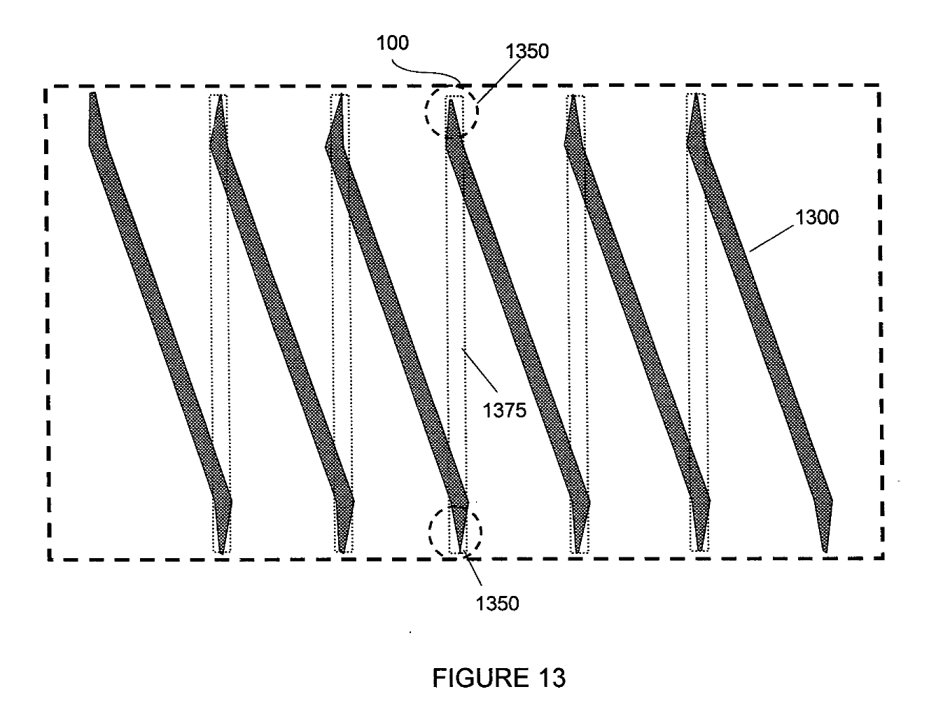

As illustrated in FIG. 13, a substrate 100 has formed thereon conductive traces 1300, composed of film coatings of metal or any thin pliable conductive material. The traces 1300 are formed so as to create a single spiraling resonance coil that will be used in forming a LC circuit that is tuned to the desired frequency of magnetic resonance imaging or other desired frequency. It is noted that the traces 1300 may also be formed so as to create a resonance coil that will be used in forming a RLC circuit that is tuned to the desired frequency of magnetic resonance imaging or other desired frequency.

In this embodiment, the “resistor” is the “conductive” material or conductive traces 1300. The resistor value is controlled by the dimensions of the conductor as well as the material selected for the conductor. Also, the material for the conductor may vary along the length of the tracing forming the inductor, thereby providing a resistive parameter to the circuit.

The degree of resonance or ‘Q’ of either the formed LC or formed RLC circuit is a degree of resonance at the Lamar frequency of the magnetic resonance imaging system to permit clinically effective imaging inside the lumen of the stent.

The capacitance is realized by the capacitance by the overlapping of the end portions of the traces 1300 in region 1350 when the substrate 100 is wrapped into a substantially cylinder shape to form a sleeve. The end portions of the traces 1300, as illustrated in FIG. 13, are formed so that the end portions are aligned as illustrated by dashed box 1375 and have a shape that enables a non-linear variability in the capacitance as he substrate 100 becomes tighter (contracts) or is loosened (expands).

The traces 1300 are insulated by an insulative dielectric material (not shown) so that when the end portions of the traces 1300 in region 1350 overlap, due to the wrapping of the substrate 100, the overlapped portions of the traces 1300 form a capacitor. The capacitance of the trace formed capacitor in region 1350 is variable as the wrapping of the substrate 100 becomes tighter (contracts) or is loosened (expands).

As noted above, the stent must enable imaging and visualization of the inner lumen of an implanted stent by means of a magnetic resonance imaging technique, thus the stent must have an associated resonance circuit that is tuned to the desired frequency of magnetic resonance. The substrate sleeve of FIG. 13 provides the resonance circuit that is tuned to the desired frequency of magnetic resonance independent of the stent.

To be in resonance, the substrate sleeve of FIG. 13 must include an LC or RLC circuit that is tuned to the desired frequency of magnetic resonance or other desired frequency.

In this embodiment, the traces are formed to create the inductive properties and the overlapping of the traces, when the sleeve is wrapped, creates the capacitive properties. Again, it is noted that a resistive value related to the dimensions of the conductor as well as the material selected for the conductor may be included in the resonance circuit of the substrate sleeve.

It is noted that as the wrapping of the substrate 100 becomes tighter (contracts), the overall inductance of the resonance circuit of the substrate sleeve decreases, but the overall capacitance of the resonance circuit of the substrate sleeve non-linearly increases because the area of the overlapping trace portions becomes greater, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that as the wrapping of the substrate 100 becomes looser (expands), the overall inductance of the resonance circuit of the substrate sleeve increases, but the overall capacitance of the resonance circuit of the substrate sleeve non-linearly decreases because the area of the overlapping trace portions becomes lesser, thereby substantially maintaining resonance with the desired frequency of magnetic resonance imaging or other desired frequency.

It is noted that the combination of the increasing of the overall inductance of the resonance circuit of the substrate sleeve and the decreasing of the overall capacitance of the resonance circuit of the substrate sleeve may also enable the maintaining of resonance within a desired bandwidth, which may or may not be in resonance with the magnetic resonance imaging scanner.

The substrate 100 may be a biodegradable substrate that essentially decomposes once the stent is positioned in the body. It is further noted that the substrate 100 may be thermally degradable, chemically degradable, and/or optically degradable. The substrate 100 may also include drugs or medical agents that are therapeutically released upon the decomposition of the substrate. Lastly, the substrate 100 is expandable without resulting in breakage. It is noted that the substrate or support web 100, may be biodegradable and may have adhesive properties useful during manufacture and implantation; however, after biodegradation, the applied conductive traces 1300 retain an electrically insulating coating or sheath that prevents unwanted shorting even under repeated flexing of the stent/circuit device in the body.

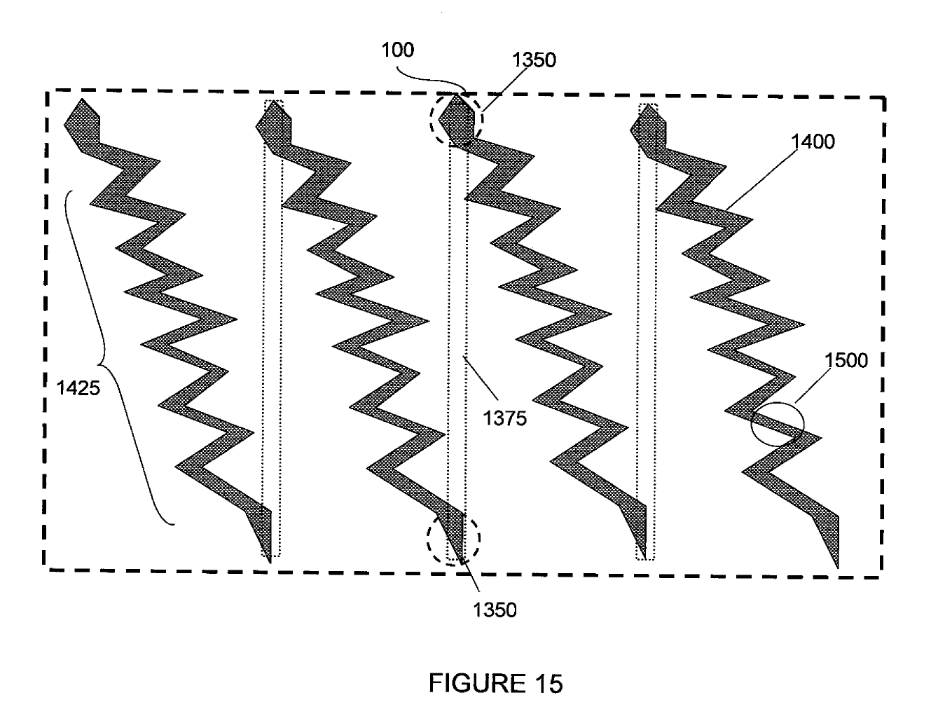

As illustrated in FIG. 15, a substrate 100 has formed thereon conductive traces 1400, composed of film coatings of metal or any thin pliable conductive material. The traces 1400 have zig-zag shape portion 1425 to prevent circuit breakage either during crimping or re-expansion.

The traces 1400 are formed so as to create a single spiraling resonance coil that will be used in forming a LC circuit that is tuned to the desired frequency of magnetic resonance imaging or other desired frequency It is noted that the traces 1400 may also be formed so as to create a resonance coil that will be used in forming a RLC circuit that is tuned to the desired frequency of magnetic resonance imaging or other desired frequency.

In this embodiment, the “resistor” is the “conductive” material or conductive traces 1400. The resistor value is controlled by the dimensions of the conductor as well as the material selected for the conductor. Also, the material for the conductor may vary along the length of the tracing forming the inductor, thereby providing a resistive parameter to the circuit.

The degree of resonance or ‘Q’ of either the formed LC or formed RLC circuit is a degree of resonance at the Lamar frequency of the magnetic resonance imaging system to permit clinically effective imaging inside the lumen of the stent.

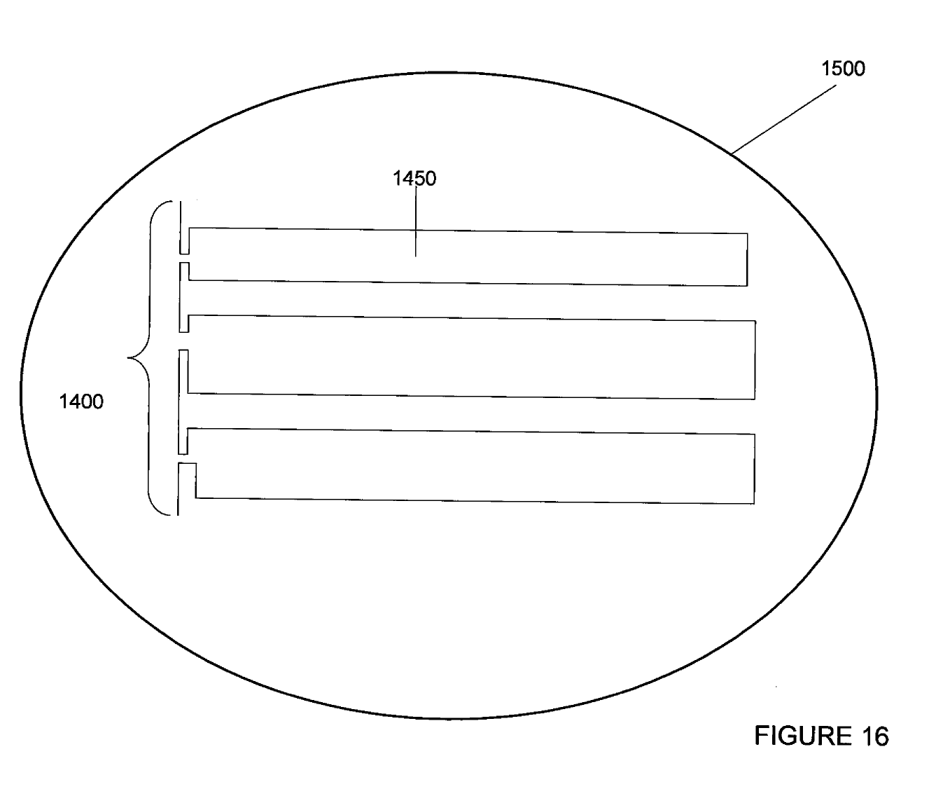

In an optional embodiment, as illustrated in FIG. 16, the traces 1400 may be formed to create sub-coils 1450. The sub-coils 1450 are formed by a finer meandering of the traces 1400 on the substrate 100. These sub-coils 1450 may be formed in any of the various embodiments discussed above.

The capacitance is realized by the capacitance by the overlapping of the end portions of the traces 1400 in region 1350 when the substrate 100 is wrapped into a substantially cylinder shape to form a sleeve. The end portions of the traces 1400, as illustrated in FIG. 13, are formed so that the end portions are aligned as illustrated by dashed box 1375 and have a shape that enables a non-linear variability in the capacitance as the substrate 100 becomes tighter (contracts) or is loosened (expands).

The traces 1400 are insulated by an insulative dielectric material (not shown) so that when the end portions of the traces 1400 in region 1350 overlap, due to the wrapping of the substrate 100, the overlapped portions of the traces 1400 form a capacitor. The capacitance of the trace formed capacitor in region 1350 is variable as the wrapping of the substrate 100 becomes tighter (contracts) or is loosened (expands).

As noted above, the stent must enable imaging and visualization of the inner lumen of an implanted stent by means of a magnetic resonance imaging technique, thus the stent must have an associated resonance circuit that is tuned to the desired frequency of magnetic resonance. The substrate sleeve of FIG. 13 provides the resonance circuit that is tuned to the desired frequency of magnetic resonance independent of the stent. The resonance circuit of FIG. 13 can also be designed to complement the resonance frequency of an implanted stent so that the combination of the resonance circuit, the implanted stent, and surrounding environmental conditions has an effective resonance frequency that is substantially equal to the operating frequency of the magnetic resonance imaging scanner. It is noted that the resonance circuit can be designed to complement the resonance frequency of any implanted device having a lumen to be imaged so that the combination of the resonance circuit, the implanted device, and surrounding environmental conditions has an effective resonance frequency that is substantially equal to the operating frequency of the magnetic resonance imaging scanner.

To be in resonance, the substrate sleeve of FIG. 13 must include an LC or RLC circuit that is tuned to the desired frequency of magnetic resonance or other desired frequency.

In this embodiment, the traces are formed to create the inductive properties and the overlapping of the traces, when the sleeve is wrapped, creates the capacitive properties. Again, it is noted that a resistive value related to the dimensions of the conductor as well as the material selected for the conductor may be included in the resonance circuit of the substrate sleeve.

It is noted that as the wrapping of the substrate 100 becomes tighter (contracts), the overall inductance of the resonance circuit of the substrate sleeve decreases, but the overall capacitance of the resonance circuit of the substrate sleeve non-linearly increases because the area of the overlapping trace portions becomes greater, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that as the wrapping of the substrate 100 becomes looser (expands), the overall inductance of the resonance circuit of the substrate sleeve increases, but the overall capacitance of the resonance circuit of the substrate sleeve non-linearly decreases because the area of the overlapping trace portions becomes lesser, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

The substrate 100 may be a biodegradable substrate that essentially decomposes once the stent is positioned in the body. It is further noted that the substrate 100 may be thermally degradable, chemically degradable, and/or optically degradable. The substrate 100 may also include drugs or medical agents that are therapeutically released upon the decomposition of the substrate. Lastly, the substrate 100 is expandable without resulting in breakage. It is noted that the substrate or support web 100, may be biodegradable and may have adhesive properties useful during manufacture and implantation; however, after biodegradation, the applied conductive traces 1400 retain an electrically insulating coating or sheath that prevents unwanted shorting even under repeated flexing of the stent/circuit device in the body.

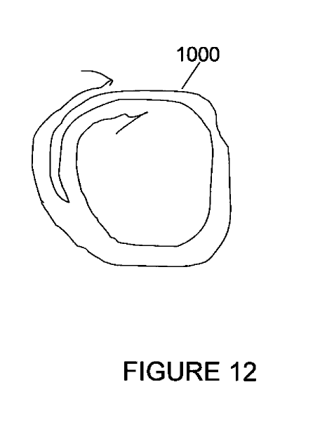

As illustrated in FIG. 12, a substrate 1000 is formed such that it wraps back into itself. This substrate includes conductive traces composed of film coatings of metal or any thin pliable conductive material. The traces are formed so as b create a single spiraling resonance coil that will be used in forming a LC circuit that is tuned to magnetic resonance imaging parameters. It is noted that the traces may also be formed so as to create a spiraling resonance coil that will be used in forming a RLC circuit that is tuned to magnetic resonance imaging parameters. The degree of resonance or ‘Q’ of either the formed LC or formed RLC circuit is a degree of resonance at the Lamar frequency of the magnetic resonance imaging system to permit clinically effective imaging inside the lumen of the stent.

The capacitance is realized by the capacitance by the overlapping of the end portions of the traces as the substrate 1000 is wrapped back into itself to form a sleeve. In other words, the substrate 1000 includes a closed end and an open end, wherein the closed end and open end are substantially parallel with the axis of the created sleeve. In this embodiment, the closed end is positioned within the open end of the substrate 1000. It is noted that either the closed end, open end, or both ends may include members (not shown) to prevent the closed end from being positioned outside (or without) the confines of the open end and open end.

The traces are insulated by an insulative dielectric material (not shown) so that when the end portions of the traces overlap, due to the wrapping of the substrate 1000, the overlapped portions of the traces form a capacitor. The capacitance of the trace formed capacitor is variable as the wrapping of the substrate 1000 becomes tighter (contracts) or is loosened (expands).

As noted above, the stent must enable imaging and visualization of the inner lumen of an implanted stent by means of a magnetic resonance imaging technique, thus the stent must have an associated resonance circuit that is tuned to the desired frequency of magnetic resonance. The substrate sleeve of FIG. 12 provides the resonance circuit that is tuned to the desired frequency of magnetic resonance independent of the stent. The resonance circuit of FIG. 12 can also be designed to complement the resonance frequency of an implanted stent so that the combination of the resonance circuit, the implanted stent, and surrounding environmental conditions has an effective resonance frequency that is substantially equal to the operating frequency of the magnetic resonance imaging scanner. It is noted that the resonance circuit can be designed to complement the resonance frequency of any implanted device having a lumen to be imaged so that the combination of the resonance circuit, the implanted device, and surrounding environmental conditions has an effective resonance frequency that is substantially equal to the operating frequency of the magnetic resonance imaging scanner.

To be in resonance, the substrate sleeve of FIG. 12 must include an LC or RLC circuit that is tuned to the desired frequency of magnetic resonance or other desired frequency.

In this embodiment, the traces are formed to create the inductive properties and the overlapping of the traces, when the sleeve is wrapped, creates the capacitive properties. Again, it is noted that a resistive value related to the dimensions of the conductor as well as the material selected for the conductor may be included in the resonance circuit of the substrate sleeve.

It is noted that as the wrapping of the substrate 1000 becomes tighter (contracts), the overall inductance of the substrate sleeve decreases, but the overall capacitance of the substrate sleeve increases because the area of the overlapping trace portion becomes greater, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that as the wrapping of the substrate 1000 becomes looser (expands), the overall inductance of the substrate sleeve increases, but the overall capacitance of the substrate sleeve decreases because the area of the overlapping trace portions becomes lesser, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

The substrate 1000 may be a biodegradable substrate that essentially decomposes once the stent is positioned in the body. It is further noted that the substrate 1000 may be thermally degradable, chemically degradable, and/or optically degradable. The substrate 1000 may also include drugs or medical agents that are therapeutically released upon the decomposition of the substrate. Lastly, the substrate 1000 is expandable without resulting in breakage. It is noted that the substrate or support web 1000, may be biodegradable and may have adhesive properties useful during manufacture and implantation; however, after biodegradation, the applied conductive traces retain an electrically insulating coating or sheath that prevents unwanted shorting even under repeated flexing of the stent/circuit device in the body.

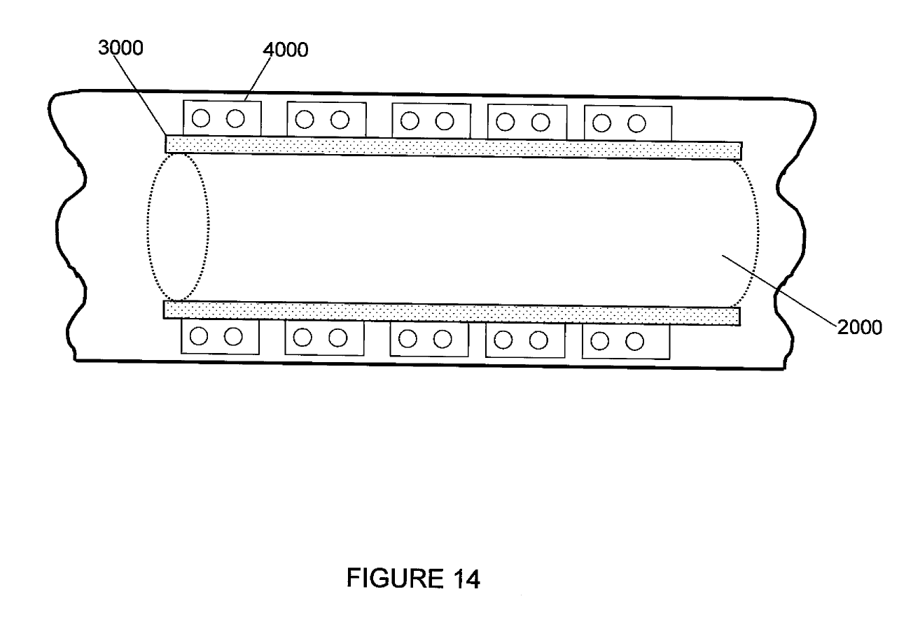

FIG. 14 illustrates a sleeve wrapped around a stent 2000. The sleeve includes a substrate 3000 has formed thereon a LC circuit 4000 that is tuned to magnetic resonance imaging parameters.

As noted above, the stent must enable imaging and visualization of the inner lumen of an implanted stent by means of a magnetic resonance imaging technique, thus the stent must have an associated resonance circuit that is tuned to the desired frequency of magnetic resonance. The substrate sleeve of FIG. 14 provides the resonance circuit that is tuned to the desired frequency of magnetic resonance independent of the stent. The resonance circuit of FIG. 14 can also be designed to complement the resonance frequency of an implanted stent so that the combination of the resonance circuit, the implanted stent, and surrounding environmental conditions has an effective resonance frequency that is substantially equal to the operating frequency of the magnetic resonance imaging scanner. It is noted that the resonance circuit can be designed to complement the resonance frequency of any implanted device having a lumen to be imaged so that the combination of the resonance circuit, the implanted device, and surrounding environmental conditions has an effective resonance frequency that is substantially equal to the operating frequency of the magnetic resonance imaging scanner.

To be in resonance, the substrate sleeve of FIG. 14 must include an LC or RLC circuit 4000 that is tuned to the desired frequency of magnetic resonance or other desired frequency.

In this embodiment, the traces are formed to create the inductive properties and the overlapping of the traces, when the sleeve is wrapped, creates the capacitive properties. Again, it is noted that a resistive value related to the dimensions of the conductor as well as the material selected for the conductor may be included in the resonance circuit of the substrate sleeve.

It is noted that as the wrapping of the substrate 3000 becomes tighter (contracts), the overall inductance of the substrate sleeve decreases, but the overall capacitance of the substrate sleeve non-linearly increases because the area of the overlapping trace portions becomes greater, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

It is also noted that as the wrapping of the substrate 3000 becomes looser (expands), the overall inductance of the substrate sleeve increases, but the overall capacitance of the substrate sleeve nonlinearly decreases because the area of the overlapping trace portions becomes lesser, thereby substantially maintaining resonance with the desired frequency of magnetic resonance imaging or other desired frequency.

It is noted that the combination of the decreasing of the overall inductance of the resonance circuit of the substrate sleeve and the increasing of the overall capacitance of the resonance circuit of the substrate sleeve may also enable the maintaining of resonance within a desired bandwidth, which may or may not be in resonance with the magnetic resonance imaging scanner.

The substrate 3000 may be a biodegradable substrate that essentially decomposes once the stent is positioned in the body. It is further noted that the substrate 3000 may be thermally degradable, chemically degradable, and/or optically degradable. The substrate 3000 may also include drugs or medical agents that are therapeutically released upon the decomposition of the substrate. Lastly, the substrate 3000 is expandable without resulting in breakage.

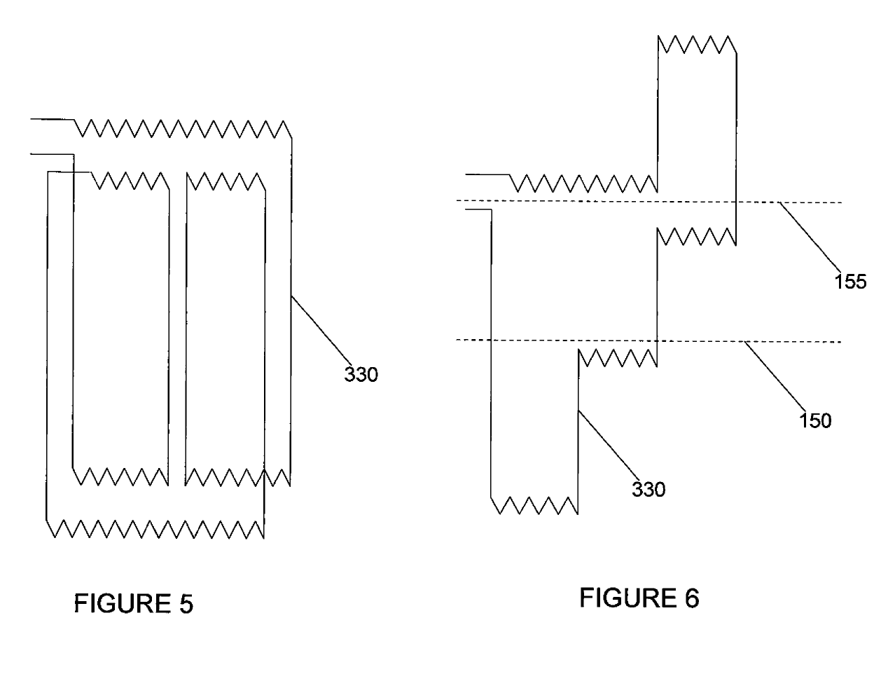

FIGS. 5 and 6 illustrate a manufacturing process for creating the sleeve substrate of the present invention. As illustrated in FIG. 6, an inductive resonance circuit is etched in foil to form traces 330. The traces 330 are folded along lines 150 and 155 to create a substantially flat resonance inductive circuit, as illustrated in FIG. 5. The folding of the etched foil enables efficient production of the traces without worry of shorting and allows the coil traces to cross over each other without short because the traces are coated in an insulative material before folding. The material may also be a dielectric, thereby providing some capacitance to the resonance circuit etched in the foil. The folding also enables the geometry of the resonance circuit topologically possible.

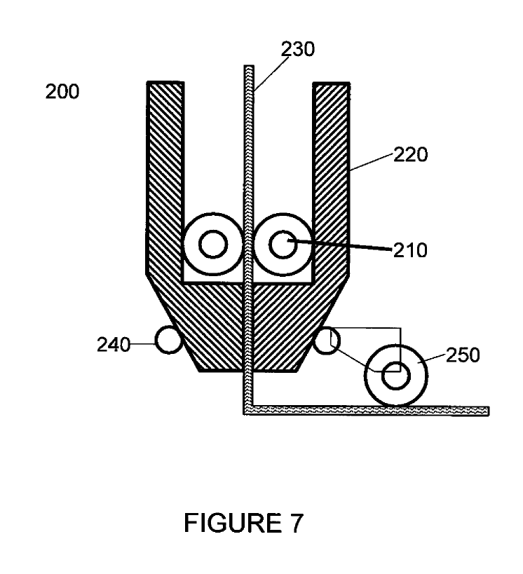

FIG. 7 illustrates another approach of manufacture. The manufacture device 200 feeds an insulated thin wire conductor 230, using drivers 210 to a substrate (not shown). The insulated thin wire conductor 230 is heated by heaters 220. The heating of the insulated thin wire conductor 230 provides an adhesive property for bonding the insulated thin wire conductor 230 to the substrate. The bond is completed by the cool roller 250 which is attached to the tool 200 by ring 240.

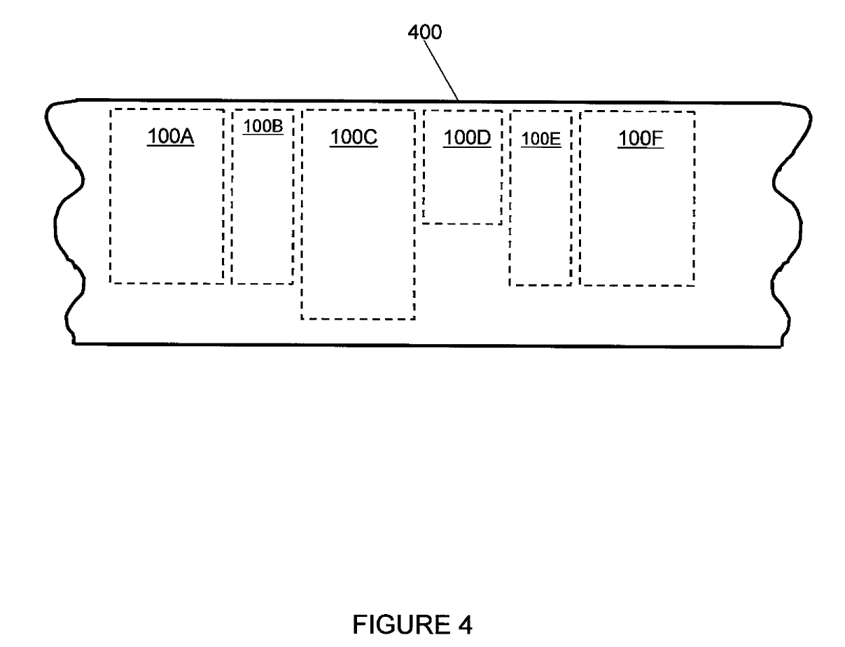

For any of the embodiments described above, the resonance circuits of FIG. 4 (100A, 100B, 100C, 100D, 100E, 100F . . . . ) may be placed upon a web 400 to provide transport. The web 400 may be a biodegradable substrate that essentially decomposes once the stent is positioned in the body. The web 400 may also include drugs or medical agents that are therapeutically released upon the decomposition of the substrate. Lastly, the web 400 is expandable without resulting in breakage. It is noted that the substrate or support web 400, may be biodegradable and may have adhesive properties useful during manufacture and implantation; however, after biodegradation, the applied resonance circuits retain an electrically insulating coating or sheath that prevents unwanted shorting even under repeated flexing of the stert/circuit device in the body It is further noted that the support web 400 may be thermally degradable, chemically degradable, and/or optically degradable.

It is noted that the end portions of the traces may have various shapes so as to provide the proper variability in the capacitance, whether it be linear variability, non-linear variability or gradual variability, etc.

It is also noted that the traces can be formed to provide variability in the inductance, whether it be linear variability, non-linear variability or gradual variability, etc.

In the various examples above, the present invention is directed to the attachment of a secondary formed structure (resonance circuit sleeve) to a primary formed structure (medical device and/or stent). This attachment of a secondary formed structure can provide imaging and visualization of the inner lumen of the primary formed structure by means of a magnetic resonance imaging technique wherein the secondary formed structure is independent of the primary formed structure's architecture. The resonance circuit can also be designed to complement the resonance frequency of an implanted primary formed structure so that the combination of the resonance circuit, the implanted primary formed structure, and surrounding environmental conditions has an effective resonance frequency that is substantially equal to the operating frequency of the magnetic resonance imaging scanner. It is noted that the resonance circuit can be designed to complement the resonance frequency of any primary formed structure having a lumen to be imaged so that the combination of the resonance circuit, the implanted primary formed structure, and surrounding environmental conditions has an effective resonance frequency that is substantially equal to the operating frequency of the magnetic resonance imaging scanner.

Moreover, the resonance circuit sleeve of the present invention provides imaging and visualization of the inner lumen of the primary formed structure (medical device and/or stent) by means of a magnetic resonance imaging technique wherein the resonance circuit sleeve of the present invention is independent of the primary formed structure's architecture.

As noted above, the resonance circuit sleeve is to be realized over an expanded stent. Initially, the sleeve is placed around an expanded stent. The sleeve may then be shrink-wrapped around the stent so that the stent is reduced in size for proper insertion into the body. The sleeve may also be crimped with the stent therein. The traces are shaped in the crimped section so as to minimize stress during crimping, shrink-wrapping, and/or and expansion.

It is noted that the substrate onto which the coil/circuit patterns are placed may initially be a cylinder. After the patterns of materials are placed upon the cylinder substrate, the cylinder is cut/slit longitudinally. By starting with a cylinder rather than a flat substrate, the material need not require the same flexibility as material need to create the sleeve created as a flat surface.

It is also noted that although the various embodiments described above refer to the utilization of the resonance circuit sleeve with a stent, the concepts of the present invention are applicable to other situations. For example, the resonance circuit sleeve of he present invention may be utilized with other devices of similar construction that are implanted in the body, such as implantable devices having conductive structures that exhibit a Faraday Cage effect and that inhibit effective internal magnetic resonance imaging. Moreover, the resonance circuit sleeve of the present invention may be utilized with vena cava filters, heart valves, and any interventional surgical device that may exhibit a Faraday Cage effect and that inhibit effective internal magnetic resonance imaging

Furthermore, it is noted that the resonance circuit sleeve can be applied to the stent before the drug eluting coating is applied. The substrate web of the resonance circuit sleeve would be dissolved prior to the drug coating. For example, the resonance circuit sleeve may have a dual insulation on the circuit; inner layer having a higher melt temperature and the outer acting as an adhesive when heated to a more modest temperature. In this example, any substrate web would be dissolved after adhesion of the resonance circuit to stent.

It is further noted that the resonance circuit may be created on a pre-formed tube rather than as a flat circuit that is wrapped to form a tube. In the various embodiments described above, adhesive may be used to help in manufacture and in retention during implantation.

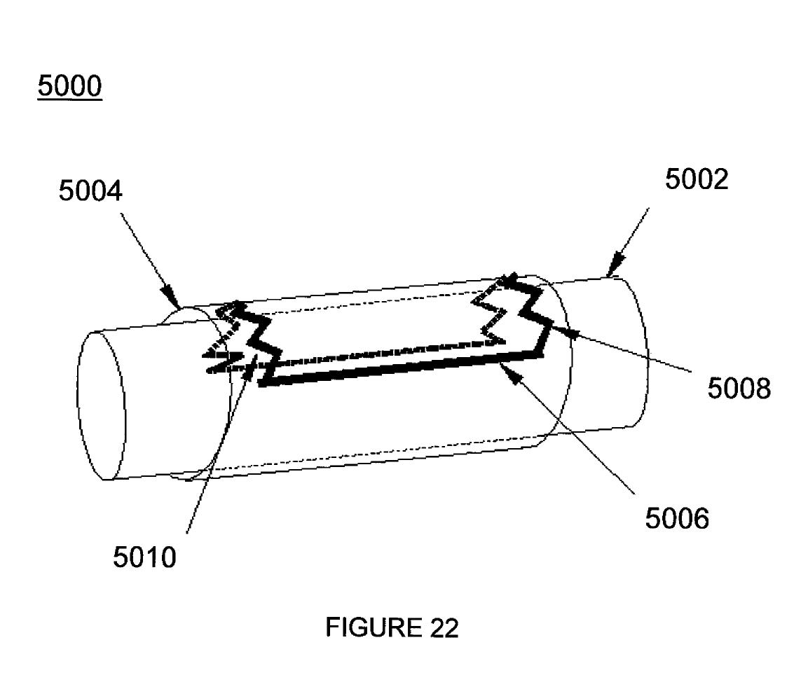

More specifically, FIG. 22 shows a stent assembly 5000 including a stent 5002 wholly or partially inserted into a cylinder membrane 5004, which may be a stent graft. The cylinder membrane 5004 has formed thereon, a circuit having one or more conductive traces 5006 forming a rectangular (or other shaped) coil and a capacitor. The capacitor is formed by overlapping two ends of the conductive trace 5006 used to form the coil. The overlapped ends of the conductive trace 5006 are separated by a dielectric material.

As illustrated in FIG. 22, the conductive traces 5006 form a rectangular shaped coil. The rectangular shaped coil has two end edges 5008 and 5010 which may have a zig-zag pattern to facilitate cylindrical radial expansion during the radial expansion of the stent 5002 and membrane 5004.

The conductive traces 5006 may form a coil having one or more coil loops. The traces 5006 may be formed side-by-side with each other or may be formed on top of each other with an electrically insulative material interposed to prevent shorting of the coil's loops.

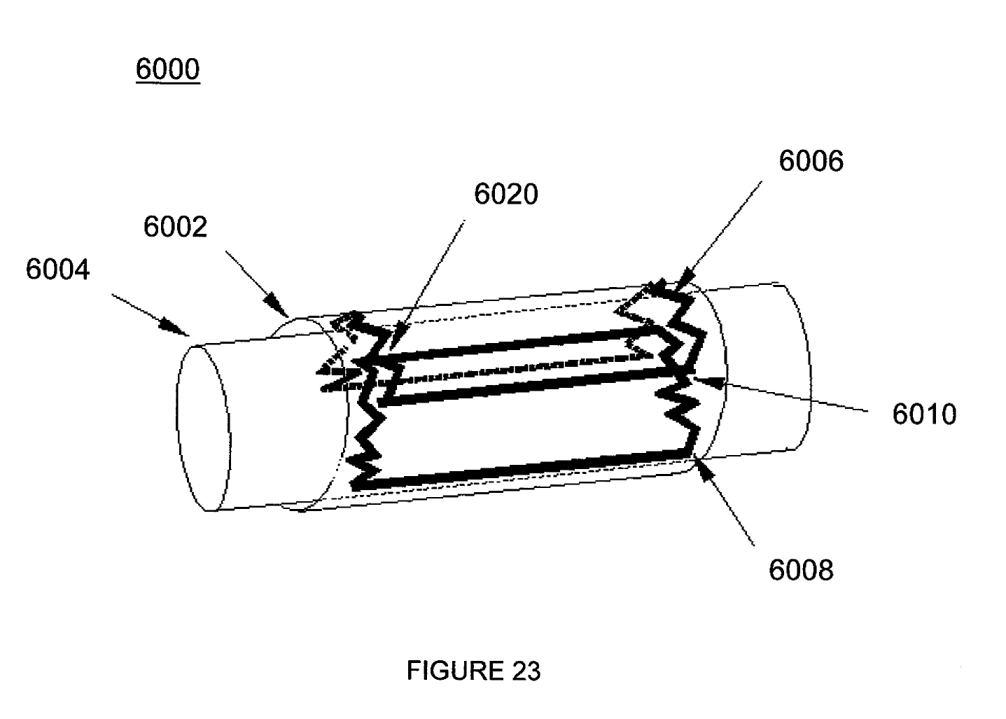

FIG. 23 illustrates a stent assembly 6000 having two resonant circuits (6006 & 6008) on a cylinder membrane 6002 around a stent 6004. The circuits (6006 & 6008) are oriented to be approximately 90 degrees to each other. At cross-over points (6010 & 6020) there is interposed an electrically insulative material.

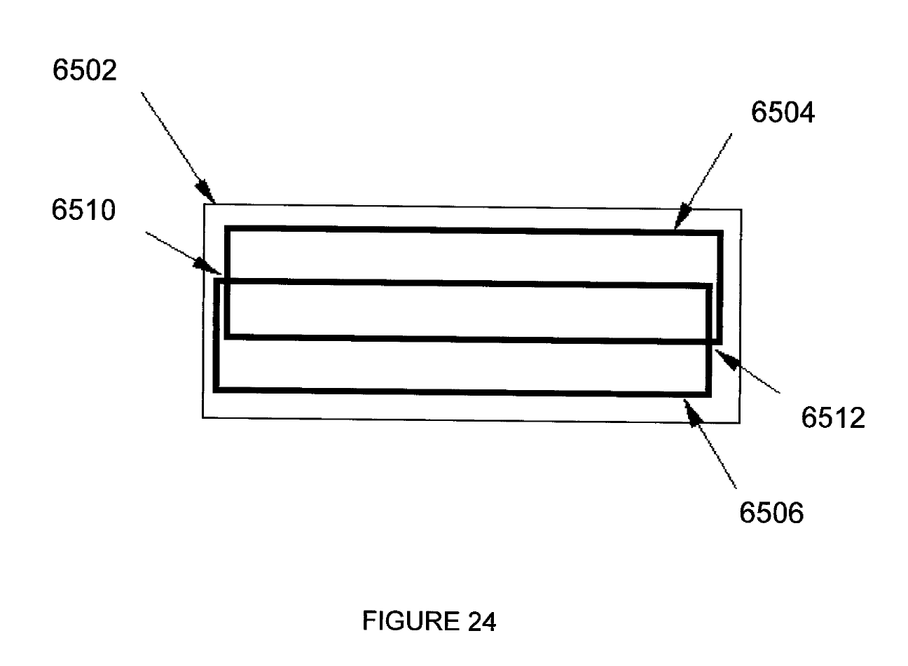

FIG. 24 illustrates a thin film substrate 6502 onto which two resonant circuits (6504 & 6506) are constructed. These circuits (6504 & 6506) each include a conductive trace to form a coil. Each coiled conductive trace has two ends (its start and stop ends). For each of the coils, overlapping the two ends of the coil trace with a dielectric interposed forms the capacitor of the circuit. The circuits (6504 & 6506) are tuned to resonate at or about the operating frequency of a magnetic resonance imaging scanner. More specifically, the circuits (6504 & 6506) are tuned so that when the circuits (6504 & 6506) are placed around a stent (or other medical device) and inserted into the body, the circuits (6504 & 6506) resonate at or near the operating frequency of the magnetic resonant imaging scanner's frequency.

As noted above, the two circuits (6504 & 6506) overlap each other (6510 & 6512). These circuits are electrically insulated from each other at these points (6510 & 6512) by placing an electrical insulative material between the conductive traces.

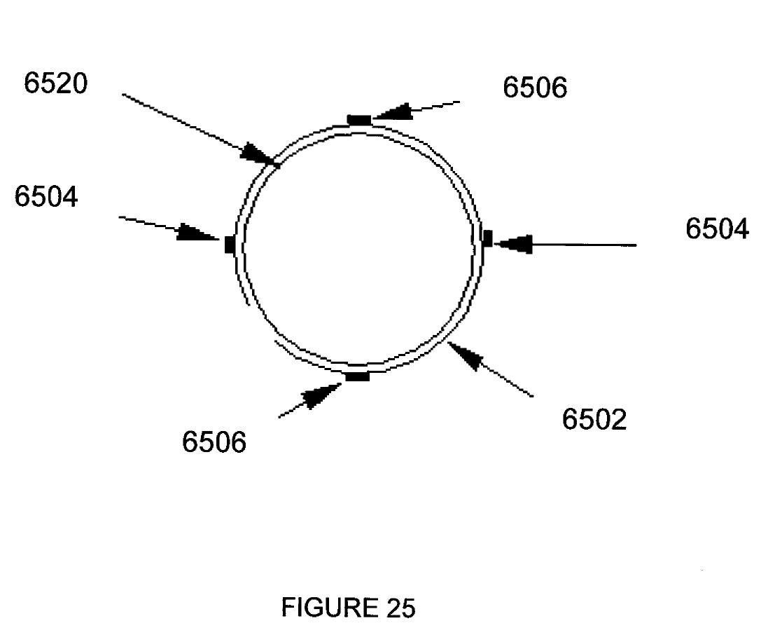

FIG. 25 illustrates the two circuits (6504 & 6506) being so positioned on the film 6502 such that when the film 6502 is wrapped around a stent 6520 the two formed coil loops are orientated at or approximately at 90 degrees to each other.

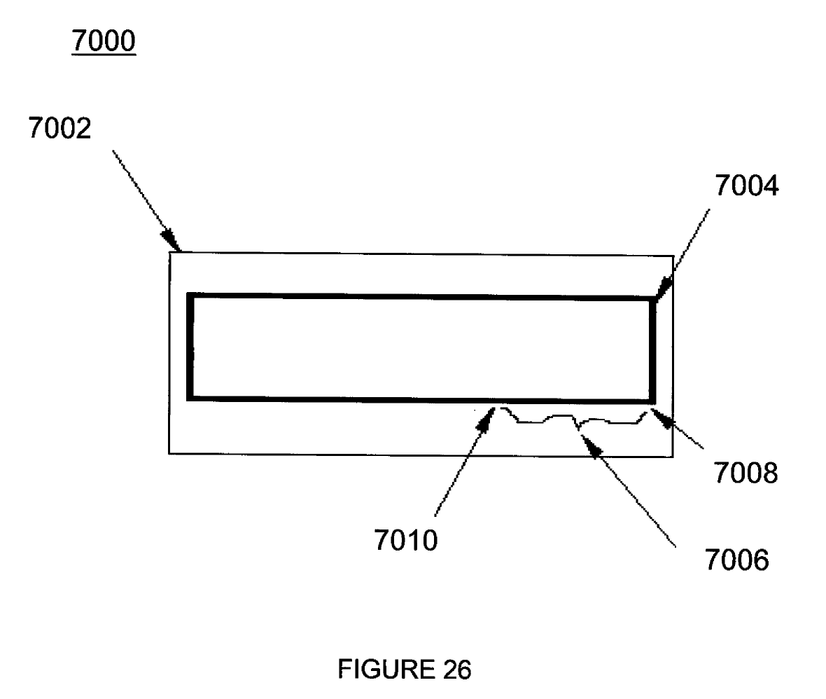

FIG. 26 illustrates the formation of a capacitor for a circuit wherein a stent circuit assembly 7000 includes a substrate 7002 onto which a conductive trace 7004 is formed. The conductive trace 7004 has a first end 7008 and a second end 7010. The two ends (7008 & 7010) of the conductive trace 7004 overlap to form a capacitor 7006.

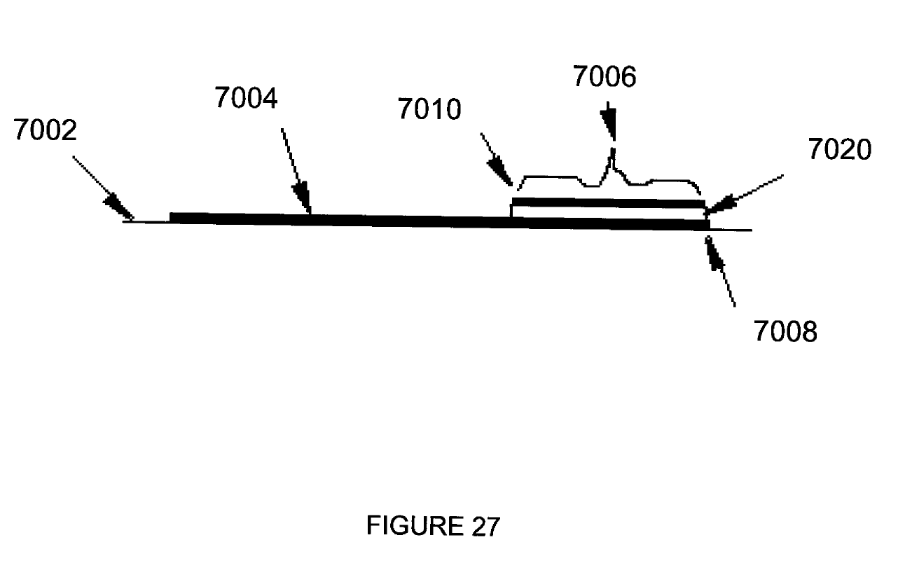

Referring to FIG. 27, which is a side view of the stent circuit assembly 7000 of FIG. 26, the conductive trace 7004 is formed on the substrate 7002. A capacitor 7006 is formed by the overlapping of the two ends (7008 & 7010) of the conductive trace 7004 with a dielectric material 7020 positioned between the two ends (7008 & 7010).

FIG. 28 illustrates a substrate 100 having a resonance coil 8500 with multiple (non-stacked) loops formed thereon. More specifically, a trace 8000 is looped to form multiple (nonstacked) loops. An area 8100, at a first end 8300 of the trace 8000 may form a capacitor when the first end 8300 of the trace 8000 overlaps a second end 8400 of the trace 8000. It is further noted that at crossover points 8200, an insulative material is interposed between the over and under traces to prevent an electrical short.

It is further noted that the resonance sleeve of the present invention may also be formed around a stent that has already been crimped into its smaller shape. It is further noted that the described substrates and/or web may the covering material for a medical device. For example, the described substrates and/or web may the covering material for a covered AAA-stent graft.

While the present invention has been particularly shown and described with references to preferred embodiments thereof, it will be understood by those skilled in the art that various changes in form and detail may be made herein without departing from the spirit and scope of the invention as defined by the appended claims.

What is claimed is:

1. A device for enabling effective magnetic resonance imaging inside a lumen of a medical device, comprising:
a substrate; and
a plurality of conductive traces formed on said substrate;
said plurality of conductive traces forming a plurality of loops to create a single spiraling coil, adjacent loops of said single spiraling coil having a non conductive material therebetween;
said single spiraling coil forming an inductive coil;
said adjacent loops of said single spiraling coil having a non-conductive material therebetween forming a capacitor;
said inductive coil and said capacitor being tuned to a frequency associated with magnetic resonance imaging.

2. The device as claimed in claim 1, wherein said inductive coil and said capacitor are tuned to an operating frequency associated with a magnetic resonance imaging scanner.

3. The device as claimed in claim 1, wherein said inductive coil and said capacitor are tuned to a harmonic of an operating frequency associated with a magnetic resonance imaging scanner.

4. The device as claimed in claim 1, wherein said inductive coil and said capacitor are tuned to a sub-harmonic of an operating frequency associated with a magnetic resonance imaging scanner.

5. The device as claimed in claim 1, wherein said substrate is biodegradable.

6. The device as claimed in claim 1, wherein said substrate is thermally degradable.

7. The device as claimed in claim 1, wherein said substrate is chemically degradable.

8. The device as claimed in claim 1, wherein said substrate is optically degradable.

9. The device as claimed in claim 1, wherein said substrate is degradable.

10. The device as claimed in claim 1, wherein said conductive traces are expandable.

11. The device as claimed in claim 1, wherein said conductive traces are expandable without damage thereto.

12. The device as claimed in claim 1, wherein said conductive traces form a pattern.

13. The device as claimed in claim 12, wherein said pattern of conductive traces is expandable.

14. The device as claimed in claim 12, wherein said pattern of conductive traces is expandable without damage thereto.

15. The device as claimed in claim 1, wherein each loop of said single spiraling coil has a distinct area.

16. The device as claimed in claim 1, wherein each loop of said single spiraling coil has a distinct shape.

17. The device as claimed in claim 1, wherein each loop of said single spiraling coil has a distinct area and a distinct shape.
